SEQUENCE LISTING (22 sequences)

SEQ ID NO: 1 - RNA, 1773 nt, Hepatitis C virus
  CDS (1)..(1773)
ucgauguccuacacauggacaggcgcccugaucacgccaugcgcugcggaggaaaccaagcugcccaucaaugcacugagcaacucuuugcuccgucaccacaacuuggucuaugcuacaacaucucgcagcgcaagccugcggcagaagaaggucaccuuugacagacugcagguccuggacgaccacuaccgggacgugcucaaggagaugaaggcgaaggcguccacaguuaaggcuaaacuucuauccguggaggaagccuguaagcugacgcccccacauucggccagaucuaaauuuggcuauggggcaaaggacguccggaaccuauccagcaaggccguuaaccacauccgcuccguguggaaggacuugcuggaagacacugagacaccaauugacaccaccaucauggcaaaaaaugagguuuucugcguccaaccagagaaggggggccgcaagccagcucgccuuaucguauucccagauuuggggguucgugugugcgagaaaauggcccuuuacgauguggucuccacccucccucaggccgugaugggcucuucauacggauuccaauacucuccuggacagcgggucgaguuccuggugaaugccuggaaagcgaagaaaugcccuaugggcuucgcauaugacacccgcuguuuugacucaacggucacugagaaugacauccguguugaggagucaaucuaccaauguugugacuuggcccccgaagccagacaggccauaaggucgcucacagagcggcuuuacaucgggggcccccugacuaauucuaaagggcagaacugcggcuaucgccggugccgcgcgagcgguguacugacgaccagcugcgguaauacccucacauguuacuugaaggccgcugcggccugucgagcugcgaagcuccaggacugcacgaugcucguaugcggagacgaccuugucguuaucugugaaagcgcggggacccaagaggacgaggcgagccuacgggccuucacggaggcuaugacuagauacucugcccccccuggggacccgcccaaaccagaauacgacuuggaguugauaacaucaugcuccuccaaugugucagucgcgcacgaugcaucuggcaaaaggguguacuaucucacccgugaccccaccaccccccuugcgcgggcugcgugggagacagcuagacacacuccagucaauuccuggcuaggcaacaucaucauguaugcgcccaccuugugggcaaggaugauccugaugacucauuucuucuccauccuucuagcucaggaacaacuugaaaaagcccuagauugucagaucuacggggccuguuacuccauugagccacuugaccuaccucagaucauucaacgacuccauggccuuagcgcauuuucacuccauaguuacucuccaggugagaucaauaggguggcuucaugccucaggaaacuugggguaccgcccuugcgagucuggagacaucgggccagaaguguccgcgcuaggcuacugucccagggggggagggcugccacuuguggcaaguaccucuucaacugggcaguaaggaccaagcucaaacucacuccaaucccggcugcgucccaguuggauuuauccagcugguucguugcugguuacagcgggggagacauauaucacagccugucucgugcccgaccccgcugguucauguggugccuacuccuacuuucuguagggguaggcaucuaucuacuccccaaccga

SEQ ID NO: 2 - protein, 591 aa, Hepatitis C virus
SMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 3 - DNA, 20 bp, Artificial
  7761R reverse primer
cgttcatcggttggggagta

SEQ ID NO: 4 - DNA, 19 bp, Artificial
  5919F forward primer
gatctcagcgacgggtctt

SEQ ID NO: 5 - DNA, 30 bp, Artificial
  L314F(c940t-F) forward primer
aggactgcacgatgttcgtatgcggagacg

SEQ ID NO: 6 - DNA, 30 bp, Artificial
  L314F(c940t-R) reverse primer
cgtctccgcatacgaacatcgtgcagtcct

SEQ ID NO: 7 - DNA, 32 bp, Artificial
  C316F(g947t-F) forward primer
gcacgatgctcgtattcggagacgaccttgtc

SEQ ID NO: 8 - DNA, 32 bp, Artificial
  C316F(g947t-R) reverse primer
gacaaggtcgtctccgaatacgagcatcgtgc

SEQ ID NO: 9 - DNA, 30 bp, Artificial
  C316S(t946a-F) forward primer
gcacgatgctcgtaagcggagacgaccttg

SEQ ID NO: 10 - DNA, 30 bp, Artificial
  C316S(t946a-R) reverse primer
caaggtcgtctccgcttacgagcatcgtgc

SEQ ID NO: 11 - DNA, 40 bp, Artificial
  S365L(c1094t-F) forward primer
gacttggagttgataacattatgctcctccaatgtgtcag

SEQ ID NO: 12 - DNA, 40 bp, Artificial
  S365L(c1094t-R) reverse primer
ctgacacattggaggagcataatgttatcaactccaagtc

SEQ ID NO: 13 - DNA, 34 bp, Artificial
  S365A(t1093g-F) forward primer
cttggagttgataacagcatgctcctccaatgtg

SEQ ID NO: 14 - DNA, 34 bp, Artificial
  S365A(t1093g-R) reverse primer
cacattggaggagcatgctgttatcaactccaag

SEQ ID NO: 15 - DNA, 38 bp, Artificial
  S365T(t1093a-F) forward primer
gacttggagttgataacaacatgctcctccaatgtgtc

SEQ ID NO: 16 - DNA, 38 bp, Artificial
  S365T(t1093a-R) reverse primer
gacacattggaggagcatgttgttatcaactccaagtc

SEQ ID NO: 17 - DNA, 34 bp, Artificial
  S368F(c7085t-F) forward primer
gataacatcatgctccttcaatgtgtcagtcgcg

SEQ ID NO: 18 - DNA, 34 bp, Artificial
  S368F(c7085t-R) reverse primer
cgcgactgacacattgaaggagcatgatgttatc

SEQ ID NO: 19 - DNA, 32 bp, Artificial
  M414T(t1241c-F) forward primer
taggcaacatcatcacgtatgcgcccaccttg

SEQ ID NO: 20 - DNA, 32 bp, Artificial
  M414T(t1241c-R) reverse primer
caaggtgggcgcatacgtgatgatgttgccta

SEQ ID NO: 21 - DNA, 32 bp, Artificial
  M414V(a1240g-F) forward primer
ctaggcaacatcatcgtgtatgcgcccacctt

SEQ ID NO: 22 - DNA, 32 bp, Artificial
  M414V(a1240g-R) reverse primer
aaggtgggcgcatacacgatgatgttgcctag

(19) United States
(12) Patent Application Publication
(10) Pub. No.: US 2010/0028922 A1
(43) Pub. Date: Feb. 4, 2010

(54) IDENTIFICATION AND CHARACTERIZATION OF HCV REPLICON VARIANTS WITH REDUCED SUSCEPTIBILITY TO BENZOFURANS, AND METHODS RELATED THERETO

(75) Inventors: Anita Y.M. HOWE, Antwerpen (BE); Rajiv CHOPRA, Andover, MA (US)

Correspondence Address: FITZPATRICK CELLA HARPER & SCINTO, 1290 Avenue of the Americas, NEW YORK, NY, 10104-3800, US

(73) Assignees: WYETH, Madison, NJ (US); VIROPHARMA INCORPORATED, Exton, PA (US)

(21) Appl. No.: 12/493,736

(22) Filed: Jun. 29, 2009

Related U.S. Application Data
(62) Division of application No. 11/842,312, filed on Aug. 21, 2007, now abandoned.
(60) Provisional application No. 60/840,353, filed on Aug. 25, 2006.

(51) Int. Cl. C12Q 1/48

(52) U.S. Cl. 435/15

(57) ABSTRACT

The present invention provides methods of decreasing the frequency of emergence, decreasing the level of resistance, and delaying the emergence of a treatment-resistant Hepatitis C viral infection, by administering to a subject, either in combination or in series, an inhibitor of the Hepatitis C RNA-dependent RNA polymerase NS5B, e.g., a benzofuran, such as 5-cyclopropyl-2-(4-fluorophenyl)-6-[(2-hydroxyethyl)(methylsulfonyl)amino]-N-methyl-1-benzofuran-3-carboxamide (HCV-796), and at least one additional anti-Hepatitis C agent, e.g., a ribavirin product or an immunomodulator, such as an interferon product. Additionally, the invention relates to methods of monitoring the course of treatment of a Hepatitis C viral infection, methods of monitoring and prognosing a Hepatitis C viral infection, and methods of identifying an individual with a decreased likelihood of responding to an anti-Hepatitis C viral therapy. These methods use the sequence and/or structure of the Hepatitis C RNA-dependent RNA polymerase NS5B to identify the emergence of a treatment-resistant Hepatitis C viral infection, particularly a benzofuran (e.g., HCV-796) treatment-resistant Hepatitis C viral infection.

This application is a divisional of U.S. application Ser. No. 11/842,312, filed Aug. 21, 2007, which claims the benefit of priority from U.S. Provisional Patent Application No. 60/840,353, filed Aug. 25, 2006, the contents of both of which are hereby incorporated by reference herein in their entirety.

### BACKGROUND OF THE INVENTION

1. Field of the Invention

The present invention relates to treatment-resistant Hepatitis C viral infections and inhibitors of Hepatitis C virus RNA-dependent RNA polymerase NS5B (RdRp), particularly benzofuran inhibitors of NS5B, more particularly 5-cyclopropyl-2-(4-fluorophenyl)-6-[(2-hydroxyethyl)(methylsulfonyl)amino]-N-methyl-1-benzofuran-3-carboxamide (HCV-796).

2. Related Background Art

Hepatitis C is a common viral infection that can lead to chronic hepatitis, cirrhosis, liver failure, and hepatocellular carcinoma. Infection with the Hepatitis C virus (HCV) leads to chronic hepatitis in at least 85% of cases, is the leading reason for liver transplantation, and is responsible for at least 10,000 deaths annually in the United States ((1997) Hepatology 26:2 S-10S).

The Hepatitis C virus is a member of the Flaviviridae family, and the genome of HCV is a single-stranded linear RNA of positive sense (Purcell (1997) Hepatology 26:11S-14S). HCV displays genetic heterogeneity; at least 6 genotypes and more than 50 subtypes have been identified (Wong and Lee (2006) Canadian Med. Assoc. J. 174:649-59).

There is no vaccine currently available to prevent HCV infection. Current therapy for HCV infection includes monotherapy treatment with interferon-α (INF-α), or a combination therapy consisting of INF-α with the nucleoside analog ribavirin (Bartenschlager (1997) Antiviral Chem. Chemo. 8:281-301). However, even with combination treatment, many patients fail to develop a sustained viral response (Wong and Lee, supra). A therapeutic response will depend on, inter alia, viral genotype, e.g., HCV genotype 1b is more resistant to IFN therapy than genotypes 2 and 3 (id.).

The HCV genome contains a number of nonstructural proteins: NS2, NS3, NS4A, NS4B, NS5A, and NS5B (Bartenschlager and Lohmann (2000) J. Gen. Virol. 81:1631-48). NS5B (RdRp) is an RNA-dependent RNA polymerase that is essential for viral replication. Previously, a proofreading property had not been identified for NS5B. The lack of proofreading mechanisms and the robust viral production (˜1×10¹² virions per day) result in high mutation rates of 10⁻⁴ to 10⁻⁵ mutations/nucleotide in HCV (Patel and Preston (1994) Proc. Natl. Acad. Sci. U.S.A. 91:549-53; Preston et al. (1988) Science 242:1168-71). As a consequence, quasi-species of viral variants have been found in HCV-infected patients (Cabot et al. (2000) J. Virol. 74:805-11; Davis (1999) Am. J. Med. 107:21 S-26S; Farci and Purcell (2000) Sem. Liver Disease 20:103-26).

NS5B RdRp is the principal catalytic enzyme for HCV replication representing a viable target for anti-HCV therapeutics (Walker and Hong (2002) Curr. Opin. Pharm. 2:534-40). Recent research efforts have led to the discovery of inhibitors that specifically target NS5B, as well as therapeutics that target other HCV viral proteins (Carroll et al. (2003) J. Biol. Chem. 278:11979-84; Dhanak et al. (2002) J. Biol. Chem. 277:38322-27; Howe et al. (2004) Antimicrobial Agents Chemo. 48:4813-21; Love et al. (2003) J. Virol. 77:7575-81; Shim et al. (2003) Antiviral Res. 58:243-51; Summa et al. (2004) J. Med. Chem. 47:14-17; Olsen et al. (2004) Antimicrobial Agents Chemo. 48:3944-53; Nguyen et al. (2003) Antimicrobial Agents Chemo. 47:3525-30; Ludmerer et al. (2005) Antimicrobial Agents Chemo. 49:2059-69; Mo et al. (2005) Antimicrobial Agents Chemo. 49: 4305-14; Lu et al. (2004) Antimicrobial Agents Chemo. 48:2260-66; U.S. Provisional Patent App. Nos. 60/735,190 and 60/735,191 (both disclosing benzofuran derivatives); U.S. Pat. No. 6,964,979 (disclosing pyranoindole derivatives); U.S. Patent Publication Nos. 2006/0063821 (disclosing arbazole and cyclopentaindole derivatives), 2004/0162318 (disclosing benzofuran derivatives), and 2004/0082643 (disclosing pyranoindole derivatives).

### SUMMARY OF THE INVENTION

Among the NS5B polymerase inhibitors reported to date, the benzofuran compound HCV-796 represents one of the most potent and selective antiviral agents both in vitro and in vivo. However, due to the high error rate that occurs during HCV replication, mutations accumulating in NS5B sometimes lead to decreased sensitivity to NS5B polymerase inhibitors. Such mutations can result in the emergence of treatment-resistant Hepatitis C viral infections. In fact, during chemotherapy, the high rates of viral replication and the high frequency of mutation currently lead to the rapid generation of drug-resistant virions. In the case of human immunodeficiency virus (HIV) and hepatitis B virus (HBV), numerous mutations have been identified in patients treated with protease inhibitors as well as nucleoside and nonnucleoside reverse transcriptase inhibitors. Emergence of resistant viruses is anticipated to be one of the largest challenges in developing effective antiviral therapies against HCV infection. Thus, there is a need to identify those mutation sites in the NS5B polymerase that result in treatment-resistant Hepatitis C viral infections. Once identified, these sites will serve as markers to monitor the course of an anti-Hepatitis C therapy for developing an increased resistance to NS5B polymerase inhibitors (e.g., benzofurans, such as HCV-796), markers to identify individuals with a decreased likelihood of responding to an anti-Hepatitis C virus therapy, and markers to monitor and prognose a Hepatitis C viral infection. This information is additionally useful to optimize second-generation Hepatitis C viral inhibitors or HCV inhibitor combinations that exhibit significantly reduced, minimal, or no susceptibility to resistance caused by mutations at these sites.

The present invention provides methods of decreasing the frequency of emergence, decreasing the level of resistance, and delaying the emergence of a treatment-resistant Hepatitis C viral infection, by administering to a subject, either in combination or in series, an inhibitor of the Hepatitis C RNA-dependent RNA polymerase NS5B, e.g., a benzofuran, such as 5-cyclopropyl-2-(4-fluorophenyl)-6-[(2-hydroxyethyl)(methylsulfonyl)amino]-N-methyl-1-benzofuran-3-carboxamide (HCV-796), and at least one additional anti-Hepatitis C agent, e.g., a ribavirin product or an immunomodulator, such as an interferon product. Additionally, the invention relates to methods of monitoring the course of treatment of a Hepatitis C viral infection, methods of monitoring and prognosing a Hepatitis C viral infection, and methods of identifying an individual with a decreased likelihood of responding to an anti-Hepatitis C viral therapy. The present invention also provides useful information and methods related to optimizing second-generation anti-Hepatitis C agents, e.g., optimizing identification and chemical synthesis of second-generation anti-Hepatitis C agents, for treating, e.g., a benzofuran treatment-resistant Hepatitis C viral infection in a subject.

Thus, in at least one embodiment, the invention provides a method of decreasing the frequency of emergence of a treatment-resistant Hepatitis C viral infection, comprising administering a benzofuran inhibitor of a Hepatitis C virus in combination with at least one additional anti-Hepatitis C virus agent to a subject in need thereof. In at least one other embodiment, the invention provides a method of delaying the emergence of a treatment-resistant Hepatitis C viral infection, comprising administering a benzofuran inhibitor of a Hepatitis C virus in combination with at least one additional anti-Hepatitis C virus agent to a subject in need thereof. In at least one other embodiment, the invention provides a method of decreasing the level of resistance of a treatment-resistant Hepatitis C viral infection, comprising administering a benzofuran inhibitor of a Hepatitis C virus in combination with at least one additional anti-Hepatitis C virus agent to a subject in need thereof. In some embodiments, the at least one additional anti-Hepatitis C virus agent is an immunomodulator and/or a ribavirin product. In some embodiments, the benzofuran inhibitor of a Hepatitis C virus is HCV-796.

In at least one embodiment, the invention provides a method of decreasing the emergence of an HCV-796-resistant Hepatitis C viral infection, comprising administering HCV-796 in combination with at least one additional anti-Hepatitis C virus agent to a subject in need thereof. In at least one other embodiment, the invention provides a method of decreasing the emergence of an HCV-796-resistant Hepatitis C viral infection, comprising administering HCV-796 either before or after administration of at least one additional anti-Hepatitis C virus agent to a subject in need thereof. In some embodiments, the at least one additional anti-Hepatitis C virus agent is an immunomodulator and/or a ribavirin product.

In at least one embodiment, the invention provides a method of identifying an individual with a decreased likelihood of responding to an anti-Hepatitis C viral therapy, comprising: determining the amino acid sequence or structure of the HCV-796 binding pocket of the Hepatitis C RNA-dependent RNA polymerase NS5B in a sample from the individual at a first time point; and determining the amino acid sequence or structure of the HCV-796 binding pocket of the Hepatitis C RNA-dependent RNA polymerase NS5B in a sample from the individual at a second time point, wherein a change in the amino acid sequence or structure of the HCV-796 binding pocket of the Hepatitis C RNA-dependent RNA polymerase NS5B in the sample from the individual at the second time point, in comparison to the amino acid sequence or structure of the HCV-796 binding pocket of the Hepatitis C RNA-dependent RNA polymerase NS5B from the individual at the first time point, indicates a decreased likelihood that the individual will respond to an anti-Hepatitis C viral therapy.

In at least one embodiment, the invention provides a method of identifying an individual with a decreased likelihood of responding to an anti-Hepatitis C viral therapy, comprising: determining the amino acid sequence or structure of the HCV-796 binding pocket of the Hepatitis C RNA-dependent RNA polymerase NS5B in a sample from the individual; and comparing the amino acid sequence or structure of the HCV-796 binding pocket of the Hepatitis C RNA-dependent RNA polymerase NS5B in the sample from the individual to the amino acid sequence or structure of the HCV-796 binding pocket of the Hepatitis C RNA-dependent RNA polymerase NS5B in a reference sample, wherein a change in the amino acid sequence or structure of the HCV-796 binding pocket of the Hepatitis C RNA-dependent RNA polymerase NS5B in the sample from the individual, in comparison to the amino acid sequence or structure of the HCV-796 binding pocket of the Hepatitis C RNA-dependent RNA polymerase NS5B in the reference sample, indicates a decreased likelihood that the individual will respond to an anti-Hepatitis C viral therapy.

In at least one embodiment, the invention provides a method for monitoring, diagnosing, or prognosing a treatment-resistant Hepatitis C viral infection in a subject, comprising: determining the amino acid sequence or structure of a benzofuran-binding pocket of the Hepatitis C RNA-dependent RNA polymerase NS5B in a sample from the subject; administering a benzofuran compound to the subject; and determining the amino acid sequence or structure of the benzofuran binding pocket of the Hepatitis C RNA-dependent RNA polymerase NS5B in a sample from the subject following administration of the benzofuran to the subject, wherein a change in the amino acid sequence or structure of the benzofuran binding pocket of the Hepatitis C RNA-dependent RNA polymerase NS5B in a sample from the subject following administration of the benzofuran, in comparison to the amino acid sequence or structure of the benzofuran binding pocket of the Hepatitis C RNA-dependent RNA polymerase NS5B in a sample from the subject prior to administration of the benzofuran, provides a negative indication of the effect of the treatment of the Hepatitis C viral infection in the subject.

In at least one embodiment, the invention provides a method for monitoring the course of treatment of a Hepatitis C viral infection in a subject, comprising: determining the amino acid sequence or structure of the HCV-796 binding pocket of the Hepatitis C RNA-dependent RNA polymerase NS5B in a sample from the subject; administering HCV-796 to the subject; and determining the amino acid sequence or structure of the HCV-796 binding pocket of the Hepatitis C RNA-dependent RNA polymerase NS5B in a sample from the subject following administration of HCV-796 to the subject, wherein a change in the amino acid sequence or structure of the HCV-796 binding pocket of the Hepatitis C RNA-dependent RNA polymerase NS5B in a sample from the subject following administration of HCV-796, in comparison to the amino acid sequence or structure of the HCV-796 binding pocket of the Hepatitis C RNA-dependent RNA polymerase NS5B in a sample from the subject prior to administration of HCV-796, provides a negative indication of the effect of the treatment of the Hepatitis C viral infection in the subject.

In at least one embodiment, the invention provides a method for monitoring the course of treatment of a Hepatitis C viral infection in a subject, comprising: determining the amino acid sequence or structure of the HCV-796 binding pocket of the Hepatitis C RNA-dependent RNA polymerase NS5B in a sample from the subject; administering HCV-796 and at least one additional anti-Hepatitis C agent to the subject; and determining the amino acid sequence or structure of the HCV-796 binding pocket of the Hepatitis C RNA-dependent RNA polymerase NS5B in a sample from the subject following administration of HCV-796 and at least one additional anti-Hepatitis C agent to the subject, wherein a change in the amino acid sequence or structure of the HCV-796 binding pocket of the Hepatitis C RNA-dependent RNA polymerase NS5B in a sample from the subject following administration of HCV-796 and at least one additional anti-Hepatitis C agent, in comparison to the amino acid sequence or structure of the HCV-796 binding pocket of the Hepatitis C RNA-dependent RNA polymerase NS5B in a sample from the subject prior to administration of HCV-796 and at least one additional anti-Hepatitis C agent, provides a negative indication of the effect of the treatment of the Hepatitis C viral infection in the subject. In some embodiments, the at least one additional anti-Hepatitis C virus agent is an immunomodulator and/or a ribavirin product.

In at least one embodiment, the invention provides a method for prognosing the development of a treatment-resistant Hepatitis C viral infection in a subject, comprising: determining the amino acid sequence or structure of the HCV-796 binding pocket of the Hepatitis C RNA-dependent RNA polymerase NS5B in a sample from the subject at a first time point; and determining the amino acid sequence or structure of the HCV-796 binding pocket of the Hepatitis C RNA-dependent RNA polymerase NS5B in a sample from the subject at a second time point, wherein a change in the amino acid sequence or structure of the HCV-796 binding pocket of the Hepatitis C RNA-dependent RNA polymerase NS5B in the sample from the subject at the second time point, in comparison to the amino acid sequence or structure of the HCV-796 binding pocket of the Hepatitis C RNA-dependent RNA polymerase NS5B from the subject at the first time point, indicates an increased likelihood that the subject will develop a treatment-resistant Hepatitis C viral infection.

In at least one embodiment, the invention provides a method for prognosing the development of a treatment-resistant Hepatitis C viral infection in a subject, comprising: determining the amino acid sequence or structure of the HCV-796 binding pocket of the Hepatitis C RNA-dependent RNA polymerase NS5B in a sample from the subject; and comparing the amino acid sequence or structure of the HCV-796 binding pocket of the Hepatitis C RNA-dependent RNA polymerase NS5B in the sample from the subject to the amino acid sequence or structure of the HCV-796 binding pocket of the Hepatitis C RNA-dependent RNA polymerase NS5B in a reference sample, wherein a change in the amino acid sequence or structure of the HCV-796 binding pocket of the Hepatitis C RNA-dependent RNA polymerase NS5B in the sample from the subject, in comparison to the amino acid sequence or structure of the HCV-796 binding pocket of the Hepatitis C RNA-dependent RNA polymerase NS5B in the reference sample, indicates an increased likelihood that the subject will develop a treatment-resistant Hepatitis C viral infection.

In at least one embodiment, the invention provides a method for monitoring a Hepatitis C viral infection in a subject, comprising: determining the amino acid sequence or structure of the HCV-796 binding pocket of the Hepatitis C RNA-dependent RNA polymerase NS5B in a sample from the subject at a first time point; and determining the amino acid sequence or structure of the HCV-796 binding pocket of the Hepatitis C RNA-dependent RNA polymerase NS5B in a sample from the subject at a second time point, wherein a change in the amino acid sequence or structure of the HCV-796 binding pocket of the Hepatitis C RNA-dependent RNA polymerase NS5B in the sample from the subject at the second time point, in comparison to the amino acid sequence or structure of the HCV-796 binding pocket of the Hepatitis C RNA-dependent RNA polymerase NS5B from the subject at the first time point, provides an indication that the Hepatitis C viral infection has changed in severity.

In at least one embodiment, the invention provides a method for monitoring a Hepatitis C viral infection in a subject, comprising: determining the amino acid sequence or structure of the HCV-796 binding pocket of the Hepatitis C RNA-dependent RNA polymerase NS5B in a sample from the subject; and comparing the amino acid sequence or structure of the HCV-796 binding pocket of the Hepatitis C RNA-dependent RNA polymerase NS5B in the sample from the subject to the amino acid sequence or structure of the HCV-796 binding pocket of the Hepatitis C RNA-dependent RNA polymerase NS5B in a reference sample, wherein a change in the amino acid sequence or structure of the HCV-796 binding pocket of the Hepatitis C RNA-dependent RNA polymerase NS5B in the sample from the subject, in comparison to the amino acid sequence or structure of the HCV-796 binding pocket of the Hepatitis C RNA-dependent RNA polymerase NS5B in the reference sample, provides an indication that the Hepatitis C viral infection has changed in severity.

In at least one embodiment, the invention provides a method for diagnosing the development of a treatment-resistant Hepatitis C viral infection in a subject, comprising: determining the amino acid sequence or structure of the HCV-796 binding pocket of the Hepatitis C RNA-dependent RNA polymerase NS5B in a sample from the subject at a first time point; and determining the amino acid sequence or structure of the HCV-796 binding pocket of the Hepatitis C RNA-dependent RNA polymerase NS5B in a sample from the subject at a second time point, wherein a change in the amino acid sequence or structure of the HCV-796 binding pocket of the Hepatitis C RNA-dependent RNA polymerase NS5B in the sample from the subject at the second time point, in comparison to the amino acid sequence or structure of the HCV-796 binding pocket of the Hepatitis C RNA-dependent RNA polymerase NS5B from the subject at the first time point, indicates an increased likelihood that the subject has developed or will develop a treatment-resistant Hepatitis C viral infection.

In at least one embodiment, the invention provides a method for diagnosing the development of a treatment-resistant Hepatitis C viral infection in a subject, comprising: determining the amino acid sequence or structure of the HCV-796 binding pocket of the Hepatitis C RNA-dependent RNA polymerase NS5B in a sample from the subject; and comparing the amino acid sequence or structure of the HCV-796 binding pocket of the Hepatitis C RNA-dependent RNA polymerase NS5B in the sample from the subject to the amino acid sequence or structure of the HCV-796 binding pocket of the Hepatitis C RNA-dependent RNA polymerase NS5B in a reference sample, wherein a change in the amino acid sequence or structure of the HCV-796 binding pocket of the Hepatitis C RNA-dependent RNA polymerase NS5B in the sample from the subject, in comparison to the amino acid sequence or structure of the HCV-796 binding pocket of the Hepatitis C RNA-dependent RNA polymerase NS5B in the reference sample, indicates an increased likelihood that the subject has developed or will develop a treatment-resistant Hepatitis C viral infection.

In at least some of the above embodiments provided by the invention, the HCV-796 binding pocket of the Hepatitis C RNA-dependent RNA polymerase NS5B comprises about amino acid residues 120 to 450 of the Hepatitis C RNA-dependent RNA polymerase NS5B. In some embodiments, the change in the amino acid sequence or structure of the HCV-796 binding pocket is an amino acid change selected from the group consisting of those set forth in Table 2B. In some further embodiments, changes in the amino acid sequence or structure of the HCV-796 binding pocket occur at amino acid residue 314, 316, 363, 365, 368, 414 or 445. In some further embodiments, the change in the amino acid sequence or structure of the HCV-796 binding pocket is an amino acid change selected from the group consisting of L314F, C316F, C316Y, C316S, C316N, I363V, S365A, S365T, S368F, M414I, and M414V. In some further embodiments, the Hepatitis C RNA-dependent RNA polymerase NS5B is derived from a Hepatitis C virus genotype selected from the group consisting of genotype 1a, genotype 1b, genotype 2, genotype 3, genotype 4, genotype 5, and genotype 6.

### BRIEF DESCRIPTION OF THE DRAWINGS

FIG. 1 shows multiple treatments of Clone A cells with HCV-796. Clone A cells were treated with 0.1 μM and 1 μM of HCV-796 in DMEM medium containing 2% FCS and 0.5% DMSO (without G418). The amounts of HCV RNA and rRNA in cell aliquots were estimated using a quantitative duplex TAQMAN® RT-PCR. The Y-axis represents HCV copies per μg of total cellular RNA (using rRNA as a marker for quantification). Each data point represents an average value from three replicates. (FIG. 1A) Effect of HCV-796 on HCV RNA. (FIG. 1B) Effect of HCV-796 on GAPDH RNA.

FIG. 2 shows the effect of HCV-796 on variant cells selected by HCV-796. Clone A and 796R cells were seeded at 7000 cells per well in a 96-well tissue culture dish, and treated with increasing concentrations of HCV-796 in the absence of G418. The level of HCV RNA from cultures was expressed as % HCV RNA relative to control. Each point represents an average of four replicates. The effective concentration that inhibits 50% of HCV RNA levels (EC₅₀) in the replicon-containing cells is indicated.

FIG. 3 shows the crystal structure of HCV-796-associated amino acid mutations. The protein is represented as an idealized ribbon. HCV-796 is depicted as a van der Waals surface. (FIG. 3A) Structural components of NS5B that interact with HCV-796. Structural components of NS5B that contain the resistance mutations are indicated (α-helix G, active site loop, tyrosine⁴⁴⁸ loop, α-helix M, and cysteine³⁶⁶ (serine-rich) loop). (FIG. 3B) Amino acids within the HCV-796 binding pocket of the Hepatitis C RNA-dependent RNA polymerase NS5B where substitutions were observed in the replicon variants selected by HCV-796. The methyl-acetamide group of benzofurans is indicated.

FIG. 4 shows the interactions between HCV-796 and amino acids in the HCV-796 binding pocket of the Hepatitis C RNA-dependent RNA polymerase NS5B, and how mutation C316F clashes with HCV-796. (FIG. 4A) Interactions between HCV-796 and amino acids in the HCV-796 binding pocket. HCV-796 is shown as a molecular surface. All residues within a 5 Å sphere are show as sticks. Residues that are mutated in resistant replicon strains are shown with thick bonds. (FIG. 4B) Mutation C316F clashes with HCV-796. Overlapping Van der Waals surfaces (arrows) indicate clashes between HCV-796 and a hypothetical model of resistance mutant C316F.

### DETAILED DESCRIPTION OF THE INVENTION

In the absence of an efficient infectious tissue culture for HCV, viral resistance can be studied in the HCV replicon system (Blight et al. (2000) Science 290:1972-74; Lohmann et al. (2003) J. Virol. 77:3007-19). A replicon is a subgenomic RNA that contains all essential elements and genes required for replication in the absence of structural genes. The HCV replicon also contains a foreign gene encoding a drug-selectable marker (neomycin phosphotransferase) to allow for G418 (neomycin) selection of cells that contain a functional replicon. Transfection of the HCV replicon into human hepatoma cells (Huh-7) leads to an autonomous HCV replication. The invention provides methods for the selection and characterization of replicon variants that have reduced susceptibility to HCV-796. Mapping of the amino acid changes encoded by the NS5B gene derived from the replicon variants showed that most of the mutations were located within the HCV-796 drug-binding pocket (a benzofuran-binding pocket). These mutations were shown to be responsible for the reduced susceptibility to HCV-796 in recombinant replicons and enzymes molecularly engineered with the single mutations. Additionally, the drug susceptibility of the replicon variants was evaluated in a panel of antiviral agents including pegylated interferon (PegIFN) and ribavirin (RBV). Similar susceptibility to PegIFN, RBV, and other HCV specific inhibitors was detected.

Using the sequence and/or structure of the Hepatitis C RNA-dependent RNA polymerase NS5B (hereinafter “NS5B”) or a portion of NS5B (e.g., the HCV-796 binding pocket of the Hepatitis C RNA-dependent RNA polymerase NS5B), the present invention therefore provides methods of monitoring the course of treatment of a Hepatitis C viral infection, methods of diagnosing the development of a treatment-resistant hepatitis C viral infection, methods of monitoring and prognosing a Hepatitis C viral infection, and methods of identifying an individual with a decreased likelihood of responding to an anti-Hepatitis C viral therapy.

As used herein, “Hepatitis C virus,” “Hepatitis C,” “HCV,” and the like means all genotypes of Hepatitis C (e.g., Hepatitis C 1a, 1b, 2, 3, and 4), and all subtypes and isolates thereof (see, e.g., Wong and Lee (2006) Canadian Med. Assoc. J. 174:649-59).

As used herein, “anti-Hepatitis C viral therapy” and the like means any treatment (e.g., administration of an agent) or course of treatment for HCV infection. Such therapies include administration of an agent alone, e.g., administration of an anti-Hepatitis C virus agent, such as an immunomodulator (e.g., an interferon product), or administration of agents in combination, e.g., administration of an immunomodulator either concurrently or in series with a ribavirin product. Thus either a single or sustained treatment, which may be an agent alone or in combination with at least one additional agent, is included within the meaning of “anti-Hepatitis C viral therapy” and the like.

As used herein, “anti-Hepatitis C virus agent” and the like means any agent that may be used to treat HCV infection, e.g., interferon products and other immunomodulators, ribavirin products, inhibitors of HCV enzymes, antifibrotics, etc. Such agents include those disclosed in, e.g., Carroll et al., supra; Dhanak et al., supra; Howe et al., supra; Love et al., supra; Shim et al, supra; Summa et al., supra; Olsen et al., supra; Nguyen et al., supra; Ludmerer et al., supra; Mo et al., supra; Lu et al., supra; Leyssen et al. (2000) Clin. Microbiol. Rev. 13:67-82; Oguz et al. (2005) W. J. Gastroenterol. 11:580-83; U.S. Provisional Patent App. Nos. 60/735,190 and 60/735,191; U.S. Pat. No. 6,964,979; U.S. Patent Publication Nos. 2006/0063821, 2004/0162318, 2006/0040944, 2006/0035848, 2005/0159345, 2005/0075309, 2005/0059647, 2005/0049204, 2005/0048062, 2005/0031588, 2004/0266723, 2004/0209823, 2004/0077587, 2004/0067877, 2004/0028754 and 2004/0082643; and PCT Publication No. WO 2001/032153. Examples of such agents include VIRAMIDINE® (Valeant Pharmaceuticals), MERIMEPODIB® (Vertex Pharmaceuticals), mycophenolic acid (Roche), amantadine, ACTILON® (Coley), BILN-2061 (Boehringer Ingelheim), Sch-6 (Schering), VX-950 (Vertex Pharmaceuticals), VALOPICITABINE® (Idenix Pharmaceuticals); JDK-003 (Akros Pharmaceuticals); HCV-796 (Wyeth/ViroPharma), ISIS-14803 (Isis Pharmaceuticals), ENBREL® (Wyeth);
IP-501 (Indevus Pharmaceuticals), ID-6556 (Idun Pharmaceuticals), RITUXIMAB® (Genentech), XLT-6865 (XTL), ANA-971 (Anadys), ANA-245 (Anadys) and TARVACIN® (Peregrine). Additional anti-Hepatitis C virus agents include immunomodulators, e.g., interferons (e.g., IFN α, β, and γ) and interferon products (e.g., pegylated interferons and albumin interferons), which includes both natural and recombinant or modified interferons. Examples of interferon products include, but are not limited to, ALBUFERON® (Human Genome Sciences), MULTIFERON® (Viragen), PEG-ALFACON® (Inter-Mune), OMEGA INTERFERON® (Biomedicines), INTRON® A (Schering), ROFERON® A (Roche), INFERGEN® (Amgen), PEG-INTRON® (Schering), PEGASYS® (Roche), MEDUSA INTERFERON® (Flamel Technologies), REBIF® (Ares Serono), ORAL INTERFERON ALFA® (Amarillo Biosciences), consensus interferon (CIFN) (Aladag et al. (2006) Turk. J. Gastroenterol. 17(1):35-39, and albumin-interferon-alpha (Balan et al. (2006) Antivir. Ther. 11:35-45).

As used herein, “immunomodulator” and the like means any agent capable of regulating an immune response or a portion of an immune response in a subject. Examples include, but are not limited to, agents that may regulate T-cell function (e.g., thymosin alfa-1, ZADAXIN® (Sci-Clone)), agents that enhance IFN activation of immune cells (e.g., histamine dihydrochloride, CEPLEME® (Maxim Pharmaceutical)), and interferon products.

Additional anti-Hepatitis C virus agents include antiviral agents (e.g., nucleoside analogs), such as ribavirin products. As used herein, “ribavirin product” and the like means any agent that contains ribavirin (1-β-D-ribofuranosyl-1H-1,2,4-triazole-3-carboxamide). Examples of such ribavirin products include COPEGUS® (Roche); RIBASPHERE® (Three Rivers Pharmaceuticals); VIRAZOLE® (Valeant Pharmaceuticals); and REBETOL® (Schering).

As used herein, “HCV-796” and the like means 5-cyclopropyl-2-(4-fluorophenyl)-6-[(2-hydroxyethyl)(methylsulfonyl)amino]-N-methyl-1-benzofuran-3-carboxamide, which is disclosed in, e.g., U.S. patent application Ser. No. 10/699,336 (i.e., U.S. Published Patent Application No. 2004/0162318) and U.S. Provisional Patent Application Nos. 60/735,190 and 60/735,191, the contents of which are hereby incorporated by reference herein in their entireties.

As used herein, “Hepatitis C RNA-dependent RNA polymerase NS5B,” “NS5B,” “RdRp,” and the like means the RNA-dependent RNA polymerase from any Hepatitis C virus (i.e., any HCV genotype or any subtype or isolate thereof). As used herein, “Hepatitis C RNA-dependent RNA polymerase NS5B gene” and the like means a nucleic acid that encodes a Hepatitis C RNA-dependent RNA polymerase NS5B. Polynucleotide and polypeptide sequences from various Hepatitis C genotypes and isolates (including NS5B sequences) may be found in the literature, e.g., HCV genotype 1b isolates include GenBank Accession Nos. AB049091.1; AB049088.1; ABO49101.1; AB049093.1; AF165059.1; AF165060.1; AB049099.1; AB049090.1; AB049097.1; AB049098.1; AF165062; AF165061.1; AF165049.1; AB049095.1; AJ238799.1; D50485.1; D50481.1; AB049087.1; AF165050.1; AF165057.1; AF165051.1; AF165058.1; U45476.1; AF165052.1; AF176573.1; AF139594.2; AB049089.1; D89872.1; ABO49100.1; AJ132996.1; AF165055.1; AJ238800.1; AF356827.1; AF165056.1; AB049096.1; AF165063.1; AF165064.1; AF483269.1; AF165054.1; AB049094.1; AF165053.1; D50480.1; D50483.1; D50482.1; AB049092.1; D50484.1; AB031322.1; U14286.1; U14320.1; U14284.1; U14282.1; U14287.1; U14281.1; U14283.1; U14316.1; U14318.1; U14292.1; U14290.1; AY003962.1; AY003965.1; U14291.1; AY003963.1; AY003966.1; AY003969.1; AY003977.1; AY003978.1; U14285.1; AY003967.1; AY003968.1; AY003979.1; U14289.1; AY003964.1; AY003953.1; AY003954.1; AY003959.1; U14295.1; AY003955.1; AY003956.1; AY003958.1; AY004032.1; AY003960.1; AY004034.1; AY004035.1; AY003957.1; AY003961.1; AY004033.1; U14304.1; L38356.1; L38360.1; L38372.1; AJ291248.1; AF071973.1; U14297.1; L29575.1; U14310.1; AB001040.1; AF071978.1; U14308.1; AJ291273.1; U14307.1; U14305.1; AF071962.1; AF107041.1; U14302.1; U14309.1; AF071987.1; AF071977.1; U14296.1; AF071976.1; X91416.1; AF071956.1; L23442.1; L23445.1; AJ231477.1; U14298.1; AJ231475.1; AF149894.1; AF149895.1; AJ231480.1; L23443.1; L23444.1; AJ231473.1; AJ231474.1; AJ231476.1; AY149711.1; AF149898.1; AF149901.1; AF149903.1; AF149904.1; AJ231472.1; AJ231478.1; AF149899.1; AF149900.1; AJ231469.1; AJ231471.1; AF149897.1; AF071957.1; AF149896.1; AF149902.1; AJ231470.1; AY149693.1; AY149708.1; AY149709.1; AF462285.1; AF462296.1; AF462283.1; AF462287.1; AF462295.1; AF462286.1; AF462294.1; S79604.1; AF462284.1; AF462291.1; AF462292.1; AF462288.1; and AF042790.1.

HCV genotype 1a isolates include, e.g., GenBank Accession Nos. NC_{—}004102.1; AY100171.1; AF516387.1; AY100128.1; AY100114.1; AF516389.1; AY100185.1; AF516391.1; AY100136.1; AY100132.1; AY100133.1; AY100179.1; AY100120.1; AY100135.1; AY100173.1; AY100118.1; AY100147.1; AY100176.1; AY100181.1; AY100193.1; AY100124.1; AF516388.1; AY100139.1; AY100161.1; AY100115.1; AY100122.1; AY100129.1; AY100131.1; AY100146.1; AY100166.1; AY100169.1; AY100130.1; AF516386.1; AY100183.1; AY100151.1; AY100145.1; AY100160.1; AY100172.1; AF516395.1; AY100134.1; AY100143.1; AY100144.1; AY100137.1; AY100155.1; AF516383.1; AY100119.1; AY100138.1; AY100154.1; AY100180.1; AY100162.1; AF516394.1; AY100123.1; AY100186.1; AY100152.1; AY100164.1; AY100167.1; AY100187.1; AY100141.1; AY100159.1; AY100188.1; AY100116.1; AY100121.1; AY100125.1; AY100163.1; AY100178.1; AF516392.1; AY100140.1; AY100189.1; AY100142.1; AY100149.1; AY100191.1; AY100127.1; AY100156.1; AY100184.1; AF516390.1; AF516393.1; AF516384.1; AY100168.1; AY100148.1; AY100170.1; AY100157.1; AY100174.1; AY100153.1; AY100126.1; AF516385.1; AY100117.1; AY100150.1; AY100165.1; AY100177.1; AY100182.1; AY100158.1; AF516382.1; AY100190.1; AY100175.1; AY100192.1; AF009071.1; S82227.1; AY003951.1; AY003947.1; AY003948.1; AY003949.1; AY003950.1; U14303.1; AY003952.1; AY004021.1; AY004022.1; AY004020.1; AY004019.1; AY004023.1; L38359.1; U14299.1; U14300.1; AF071960.1; AF071961.1; AF071983.11; AJ291260.1; AF071959.1; AF071963.1; AJ291247.1; Z99042.1; AF071982.1; Z99040.1; Z99043.1; AF071953.1; AF071975.1; Z99041.1; AF071984.1; AF071985.1; AF071986.1; AY149700.1; AF071965.1; AF071974.1; AF071958.1; AF071979.1; AF071981.1; AF071968.1; AF071980.1; AY149698.1; L23435.1; L23436.1; AF071966.1; AY149701.1; AY149704.1; AF071955.1; AF071964.1; AY149692.1; L23437.1; L23440.1; AJ231490.1; AJ231491.1; L23439.1; L23438.1; L23441.1; AJ231489.1; AF009073.1; AF462276.1; AF009072.1; AF462279.1; AF462278.1; AF462281.1; AF009069.1; AF462277.1; AF462280.1; AF009070.1; AF462275.1; AF462282.1; AJ231493.1; and AJ231494.1.

HCV genotype 2 isolates include, e.g., GenBank Accession Nos. AX057088.1; AX057090.1; AX057092.1; AX057094.1; D31973.1; D50409.1; AF238486.1; AB030907.1; U14293.1; U14294.1; AF238481.1; |AF238485.1; AF238484.1; U14288.1; AF238482.1; AF169002.1; AF169005.1; AF238483.1; AX057086.1; AF169003.1; AF169004.1; AY004014.1; AY004015.1; AY004016.1; AY004017.1; AY004024.1; AY004025.1; AY004026.1; AY004027.1; AY004028.1; AY004029.1; AY004030.1; AY004031.1; and AF107040.1.

HCV genotype 3 isolates include, e.g., GenBank Accession Nos. D49374.1; D17763.1; D10585.1; AF046866.1; AY100061.1; AY100033.1; AY100080.1; AY100088.1; AY100036.1; AF516379.1; AY100064.1; AY100059.1; AY100062.1; AY100065.1; AY100078.1; AF516374.1; AY100090.1; AY100042.1; AY100075.1; AF516369.1; AY100067.1; AY100045.1; AF516377.1; AY100058.1; AF516378.1; AY100026.1; AY100044.1; AY100055.1; AY100056.1; AY100092.1; AY100097.1; AY100047.1; AY100029.1; AY100028.1; AY100091.1; AF516368.1; AY100087.1; AY100052.1; AF516376.1; AY100027.1; AY100066.1; AY10101.1; AF516373.1; AF516375.1; AY100057.1; AY100032.1; AY100038.1; AY100069.1; AY100082.1; AY100083.1; AY100098.1; AF516370.1; AY100040.1; AY100093.1; AY100035.1; AY100046.1; AY100049.1; AY100050.1; AY100070.1; AY100073.1; AY100077.1; AY100085.1; AF516380.1; AY100084.1; AY100030.1; AY100109.1; AY100111.1; AY100041.1; AY100053.1; AY100095.1; AF516367.1; AF516372.1; AY100039.1; AY100043.1; AY100060.1; AY100063.1; AY100068.1; AY100072.1; AY100100.1; AY100113.1; AY100071.1; AY100076.1; AY100102.1; AY100031.1; AY100048.1; AY100108.1; AF516371.1; AY100037.1; AY100074.1; AY100096.1; AY100110.1; AY100024.1; AY100051.1; AY100079.1; AY100086.1; AY100103.1; AY100105.1; AY100107.1; AY100099.1; AF516381.1; AY100089.1; AY100094.1; AY100104.1; AY100025.1; AY100054.1; AY100081.1; AY100106.1; AY100112.1; U14315.1; U14317.1; U14313.1; AY003970.1; U14314.1; U14319.1; X91303.1; AY003975.1; AY003976.1; AY003974.1; AY004018.1; AF216791.1; U14301.1; AY003971.1; AY003973.1; AF388454.1; U14312.1; AY003972.1; and L23466.1.

HCV genotype 4 isolates include, e.g., GenBank Accession Nos. Y11604.1; AF271807.1; AF271800; AJ291255.1; AJ291293.1; AJ291258.1; AJ291291.1; AJ291282.1; AJ291284.1; AJ291263.1; AJ291286.1; AJ291272.1; AJ291275.1; AJ291271.1; AF271814.1|AF271814; AJ291254.1; AJ291289.1; AJ291288.11; AJ291249.1; L38370.1; AF388477.1; and AF271815.1.

HCV genotype 5 isolates include, e.g., GenBank Accession Nos. Y13184.1; AJ291281.1; L23472.1; and L23471.1.

HCV genotype 6 isolates include, e.g., GenBank Accession Nos. Y12083.1; L38379.1; L23475.1; and L38339.1.

As used herein, “NS5B gene product” and the like means NS5B polynucleotides and polypeptides and fragments thereof (e.g., mRNA, RNA, rRNA, cDNA, protein, peptides and fragments thereof).

As used herein, “amino acid change” and the like means a deviation from the amino acid residue at a given position in a Hepatitis C RNA-dependent RNA polymerase NS5B (e.g., an RNA-dependent RNA polymerase NS5B from Hepatitis C of genotype 1b, 2, 3, and 4) or a portion thereof (e.g., the HCV-796-binding pocket of a Hepatitis C RNA-dependent RNA polymerase NS5B) as disclosed herein or otherwise associated with HCV. The phrase “amino acid change” and the like means both single and multiple changes or differences in a Hepatitis C RNA-dependent RNA polymerase NS5B sequence or between or among sequences.

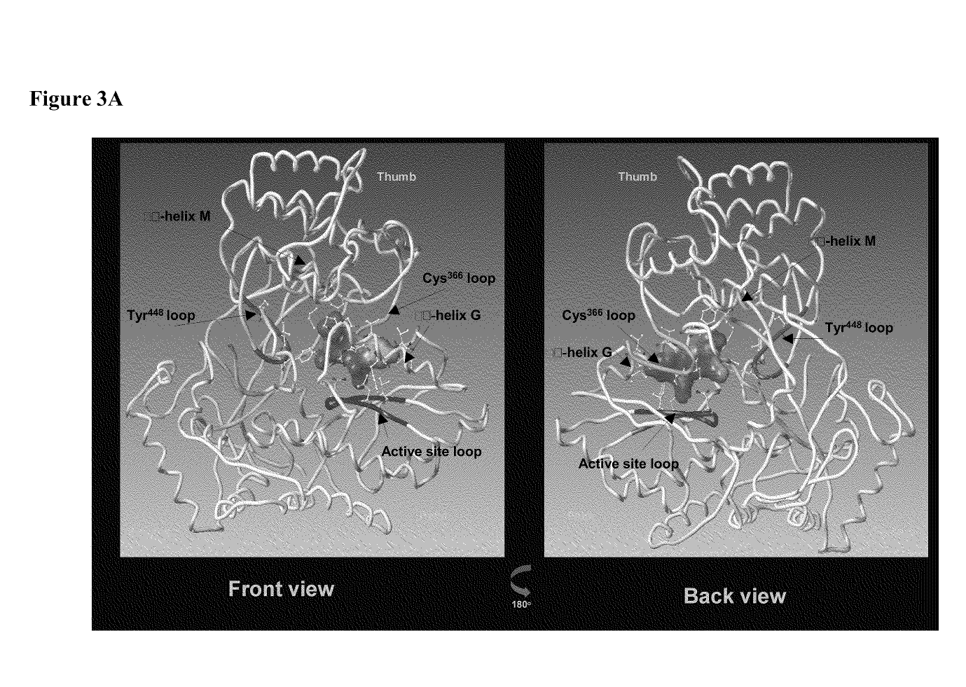

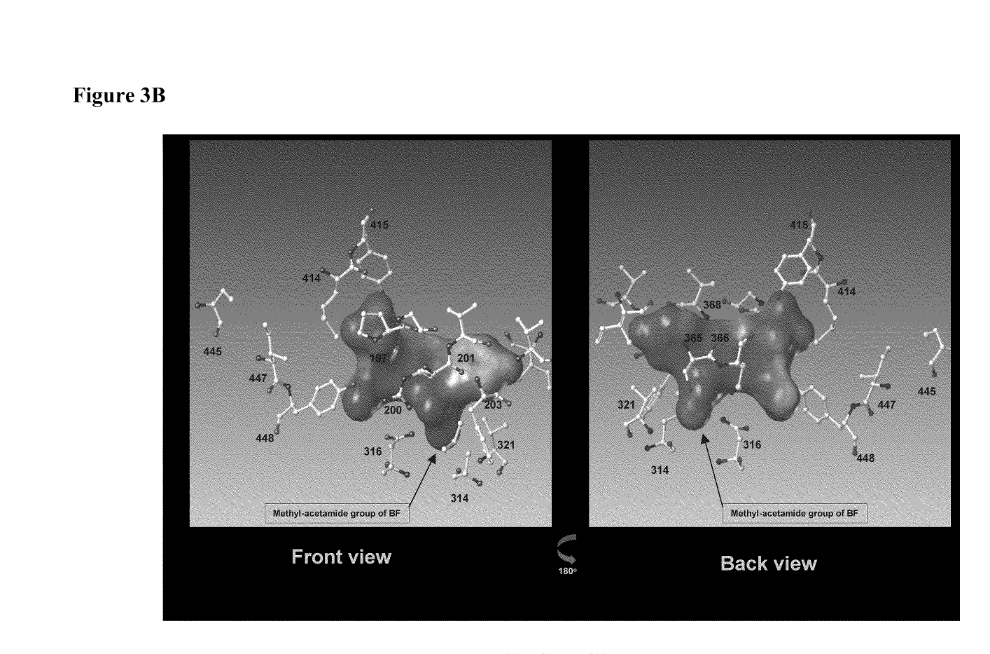

As used herein, “HCV-796 binding pocket” and the like means the portion of a Hepatitis C RNA-dependent RNA polymerase NS5B responsible for interacting with HCV-796. For example, the HCV-796-binding pocket of NS5B from HCV genotype 1b is contained within about amino acid residues 120 to 450. As shown in FIG. 3, the HCV-796 binding pocket of NS5B from HCV genotype 1b, as well other HCV genotypes, consists of five major structural elements, an active site loop, a serine-rich loop (Cys³⁶⁶ loop), the α-helix M loop, the α-helix G loop, and the Tyr⁴⁴⁸ loop.

In relation to the methods disclosed herein, determining “the amino acid sequence or structure of the HCV-796 binding pocket of the Hepatitis C RNA-dependent RNA polymerase NS5B” and the like includes, but is not limited to, (1) determining the amino acid sequence of the HCV-796 binding pocket of the Hepatitis C RNA-dependent RNA polymerase NS5B or a portion thereof; (2) determining the amino acid structure of the HCV-796 binding pocket of the Hepatitis C RNA-dependent RNA polymerase NS5B or a portion thereof; and (3) determining the nucleic acid sequence encoding the HCV-796 binding pocket of the Hepatitis C RNA-dependent RNA polymerase NS5B or a portion thereof. Such methods may employ routine nucleotide sequencing, routine protein sequencing, or antibody detection of structural changes.

In addition, the instant invention contemplates methods of decreasing the frequency of emergence, decreasing the level of resistance, and delaying the emergence of a treatment-resistant Hepatitis C viral infection, by administering to a subject, either in combination or in series, an inhibitor of the Hepatitis C RNA-dependent RNA polymerase NS5B (e.g., a benzofuran, such as HCV-796) and at least one additional anti-Hepatitis C agent (e.g., a ribavirin product or an immunomodulator, such as an interferon product). As discussed herein, administration of two or more anti-Hepatitis C virus agents (e.g., HCV-796 with an interferon product and/or a ribavirin product) may be concurrent or in series.

As described in further detail herein, exemplary agents useful to decrease the frequency of emergence, decrease the level of resistance, and delay the emergence of a treatment-resistant Hepatitis C viral infection include agents that target the Hepatitis C RNA-dependent RNA polymerase NS5B, e.g., benzofuran compounds. Such compounds are disclosed in, e.g., U.S. Provisional Patent Appln. Nos. 60/735,190 and 60/735,191, and U.S. Patent Publication No. 2004/0162318, the disclosures of which are hereby incorporated by reference herein. In one embodiment of the invention, the benzofuran compound is 5-cyclopropyl-2-(4-fluorophenyl)-6-[(2-hydroxyethyl)(methylsulfonyl)amino]-N-methyl-1-benzofuran-3-carboxamide (HCV-796). Thus, as used herein “benzofuran inhibitor of a Hepatitis C virus” and the like means a benzofuran anti-Hepatitis C virus agent.

As used herein, “delaying the emergence” and the like means postponing the development, e.g., of a Hepatitis C virus with resistance to an anti-Hepatitis C viral therapy of choice, e.g., a benzofuran anti-Hepatitis C viral therapy (such as a benzofuran-based anti-Hepatitis C viral therapy employing HCV-796). Thus, “delaying the emergence” and the like may refer to postponing the development of a treatment-resistant Hepatitis C viral infection relative to a reference sample (e.g., a reference mean or median rate of development of a treatment-resistant Hepatitis C virus in a reference population). Such postponement may be by at least 10%, 20%, 30%, 40%, 50%, 60%, 70%, 80%, 90%, or 100%, or any other method of assessing a delay of emergence of resistance known in the art.

As used herein, “decreasing the frequency of emergence” and the like means reducing the rate of occurrence, e.g., of the development of a Hepatitis C virus with resistance to an anti-Hepatitis C viral therapy of choice. Thus, “decreasing the frequency of emergence” and the like may refer to a reduction in the rate of occurrence of a treatment-resistant Hepatitis C viral infection relative to a reference sample (e.g., a reference mean or median rate of occurrence of a treatment-resistant Hepatitis C virus in a reference population). Such reduction may be by at least 10%, 20%, 30%, 40%, 50%, 60%, 70%, 80%, 90%, or 100%, or any other method of assessing a decrease of frequency of emergence of resistance known in the art.

As used herein, “decreasing the level of resistance” and the like means reducing the strength or the ability of a Hepatitis C virus to withstand an anti-Hepatitis C viral therapy. Thus, “decreasing the level of resistance” and the like may refer to a reduction in the strength or the ability of a Hepatitis C virus to withstand an anti-Hepatitis C viral therapy relative to a reference sample (e.g., a reference mean or median ability to withstand an anti-Hepatitis C viral therapy in a reference population). Such reduction may be by at least 10%, 20%, 30%, 40%, 50%, 60%, 70%, 80%, 90%, or 100%, or any other method of assessing a decrease in the level of resistance known in the art.

As used herein, “treatment-resistant Hepatitis C viral infection” and the like means a Hepatitis C viral infection that displays an abrogated response to an anti-Hepatitis C viral therapy (e.g., a delayed (or absent) response to treatment, or a lessened (i.e., abrogated) reduction in Hepatitis C viral load in response to treatment). In one embodiment of the invention the treatment-resistant Hepatitis C viral infection is a benzofuran-resistant Hepatitis C viral infection, particularly an HCV-796 resistant Hepatitis C viral infection.

Reference to a nucleotide sequence or polynucleotide as set forth herein encompasses a DNA molecule (e.g., a cDNA molecule) with the specified sequence (or a complement thereof), and encompasses an RNA molecule (e.g., an mRNA or an rRNA molecule) with the specified sequence in which U is substituted for T, unless context requires otherwise. Such polynucleotides and nucleic acids additionally include allelic variants of the disclosed polynucleotides, e.g., polynucleotides and nucleic acids of various subtypes of the Hepatitis C virus genotypes. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides. Alternatively, significant similarity exists when the nucleic acid segments will hybridize under selective hybridization conditions (e.g., highly stringent hybridization conditions) to the disclosed polynucleotides.

Such polynucleotides and nucleic acids additionally include DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least 50% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

Calculations of “homology” or “sequence identity” between two sequences are performed by means well known to those of skill in the art. For example, one general means for calculating sequence identity is described as follows. The sequences are aligned for optimal comparison purposes (e.g., gaps can be introduced in one or both of a first and a second amino acid or nucleic acid sequence for optimal alignment, and nonhomologous sequences can be disregarded for comparison purposes). In a preferred embodiment, the length of a reference sequence aligned for comparison purposes is at least 30%, preferably at least 40%, more preferably at least 50%, still more preferably at least 60%, and even more preferably at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent sequence identity between two sequences may be accomplished using a mathematical algorithm. In one exemplary embodiment, the percent identity between two amino acid sequences is determined using the Needleman and Wunsch ((1970) J. Mol. Biol. 48:444-53) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. One exemplary set of parameters is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17), which has been incorporated into the ALIGN program (version 2.0), using a PAM 120 weight residue table, a gap length penalty of 12 and a gap penalty of 4.

Anti-Hepatitis C virus agents include, e.g., polynucleotides, protein biologics, antibodies and small molecules. The term “small molecule” refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2^{nd} ed., ed. N. Rose, Wiley and Sons, New York, 14 (1995)). For example, Davis et al. (2005) Proc. Natl. Acad. Sci. USA 102:5981-86, use the phrase small molecule to indicate folates, methotrexate, and neuropeptides, while Halpin and Harbury (2004) PLos Biology 2:1022-30, use the phrase to indicate small molecule gene products, e.g., DNAs, RNAs and peptides. Examples of natural and synthesized small molecules include, but are not limited to, cholesterols, neurotransmitters, siRNAs, and various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24).

The term “pharmaceutical composition” means any composition that contains at least one therapeutically or biologically active agent (e.g., an anti-Hepatitis C virus agent(s), such as HCV-796, a ribavirin product, or an interferon product) and is suitable for administration to a subject. Pharmaceutical compositions and appropriate formulations thereof can be prepared by well-known and accepted methods of the art. See, for example, Remington: The Science and Practice of Pharmacy, 21^{st} Ed., (ed. A. R. Gennaro), Lippincott Williams & Wilkins, Baltimore, Md. (2005).

In all aspects of the invention, the Hepatitis C RNA-dependent RNA polymerase NS5B that is analyzed as part of the disclosed methods may be a variant polypeptide that differs from an NS5B sequence set forth herein. Such a variation may occur in an irrelevant site of NS5B, e.g., outside of the HCV-796-binding domain. These NS5B polypeptides are contemplated as useful in the instant methods because such methods rely on the identification of a change in sequence or structure of an NS5B polypeptide from an individual (over time, i.e., between a first and second time point, or relative to a reference sample) infected with HCV. In general, viral mutation may replace residues that form NS5B protein tertiary structure, provided that residues that perform a similar function are used. In other instances, the type of residue may be completely irrelevant if an alteration occurs in a noncritical area. Thus, the invention further utilizes NS5B variants that show substantial NS5B-type biological activity. Such variants include deletions, insertions, inversions, repeats, and type substitutions (for example, substituting one hydrophilic residue for another, but not a strongly hydrophilic residue for a strongly hydrophobic residue). Small changes or “neutral” amino acid substitutions will often have little impact on protein function (Taylor (1986) J. Theor. Biol. 119:205-18). Conservative substitutions may include, but are not limited to, replacements among the aliphatic amino acids, substitutions between amide residues, exchanges of basic residues, and replacements among the aromatic residues. Further guidance concerning which amino acid changes are likely to be phenotypically silent (i.e., are unlikely to significantly affect function) can be found in Bowie et al. (1990) Science 247:1306-10 and Zvelebil et al. (1987) J. Mol. Biol. 195:957-61.

### Methods for Monitoring the Course of Treatment of a Hepatitis C Viral Infection, Methods for Monitoring and Prognosing a Hepatitis C Viral Infection, and Methods for Diagnosing the Development of a Treatment-Resistant Hepatitis C Viral Infection

The present invention provides methods for monitoring the course of treatment of a Hepatitis C viral infection, methods for monitoring and prognosing the development of a treatment-resistant Hepatitis C viral infection, and methods for diagnosing the development of a treatment-resistant Hepatitis C viral infection, by, e.g., determining the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445 of an NS5B) in a sample from the subject, and comparing the sequence or structure of the NS5B gene product(s) or a portion(s) thereof in the sample from the subject to the sequence or structure of an NS5B gene product(s) or a portion(s) thereof in a reference sample. Alternatively, these methods may include determining a test sequence or structure of an NS5B gene product(s) or portion(s) thereof in biological sample taken from a subject at a first time point, and comparing the sequence or structure of the NS5B gene product(s) or portion(s) thereof to the sequence or structure of an NS5B gene product(s) or portion(s) thereof in a biological sample taken from a subject at a second time point.

For example, the invention provides methods of diagnosing, prognosing and monitoring, e.g., by determining changes in the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445 of an NS5B) in a sample from a subject infected with HCV. The sequence or structure of an NS5B gene product(s) or a portion(s) thereof may also be measured in a reference cell or sample of interest to produce or obtain a reference sequence or structure of NS5B, or such reference sequence or structure may be obtained through other methods, or may be generally known, by one of skill in the art. In addition, the sequence or structure of the NS5B gene product(s) or a portion(s) thereof may be obtained from a subject at a first time point and compared to the sequence or structure of the NS5B gene product(s) or portion(s) thereof from a subject at a second time point to identify the development of amino acid changes in an NS5B gene product(s) or a portion(s) thereof. These methods may be performed by, e.g., utilizing prepackaged diagnostic kits comprising at least one of a polynucleotide (or portion(s) thereof, e.g., an NS5B sequencing probe(s) or an NS5B hybridization probe(s)), or an antibody against an NS5B polypeptide (or a portion thereof), which may be conveniently used, for example, in a clinical setting.

“Diagnostic” or “diagnosing” means identifying the presence or absence of a pathologic condition, e.g., diagnosing the development of a treatment-resistant Hepatitis C viral infection in a subject. Diagnostic methods include, but are not limited to, detecting changes in the sequence or structure of the RNA-dependent RNA polymerase NS5B by determining the sequence or structure an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445 of an NS5B) in a biological sample from a subject (e.g., human or nonhuman mammal), and comparing the test sequence or structure with, e.g., a normal (or relatively normal) NS5B gene product sequence or structure (e.g., an NS5B sequence or structure from a reference sample or from the subject at an initial first time point). Although a particular diagnostic method may not provide a definitive diagnosis of the development of a treatment-resistant Hepatitis C viral infection, it suffices if the method provides a positive indication that aids in diagnosis.

The present invention also provides methods for prognosing the development of a treatment-resistant Hepatitis C viral infection in a subject by determining, for example, the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445 of an NS5B) in a biological sample from a subject (e.g., human or nonhuman mammal). “Prognostic” or “prognosing” means predicting the probable development and/or severity of a pathologic condition. Prognostic methods include determining the sequence or structure of an NS5B gene product(s) or a portion(s) thereof in a biological sample from a subject, and comparing the sequence or structure of the NS5B gene product(s) or portion(s) thereof to a prognostic sequence or structure of the NS5B gene product(s) or portion(s) thereof (e.g., an NS5B sequence or structure from a reference sample). Alternatively, prognostic methods may include determining a test sequence or structure of an NS5B gene product(s) or portion(s) thereof in a biological sample taken from a subject at a first time point, and comparing the sequence or structure of the NS5B gene product(s) or portion(s) thereof to the sequence or structure of an NS5B gene product(s) or portion(s) thereof in a biological sample taken from a subject at a second time point. Changes in a particular portion(s) (e.g., the HCV-796-binding pocket of an NS5B) or amino acid residue(s) of an NS5B gene product(s) (e.g., amino acid residues 314, 316, 363, 365, 368, 414 or 445 of an NS5B) are consistent with certain prognoses for the development of a treatment-resistant Hepatitis C viral infection.

The present invention also provides methods for monitoring a Hepatitis C viral infection in a subject by determining, for example, the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445 of an NS5B) in a biological sample from a human or nonhuman mammalian subject. Monitoring methods include determining a test sequence or structure of an NS5B gene product(s) or portion(s) thereof in a biological sample taken from a subject at a first time point, and comparing the sequence or structure of the NS5B gene product(s) or portion(s) thereof to the sequence or structure of an NS5B gene product(s) or portion(s) thereof in a biological sample taken from a subject at a second time point. Alternatively, monitoring methods may include comparing the test sequence or structure with, e.g., a normal sequence or structure of an NS5B gene product(s) or portion(s) thereof (e.g., an NS5B sequence or structure from a reference sample). A change in the sequence or structure of an NS5B gene product(s) or portion(s) thereof between the first and second time points (or between the test sample and the reference sample) indicates that the Hepatitis C viral infection has increased in severity. Such monitoring assays are also useful for evaluating the efficacy of a particular anti-Hepatitis C virus agent or an anti-Hepatitis C viral therapy in patients being treated for Hepatitis C infection, i.e., monitoring the course of treatment of a HCV infection in a subject, e.g., a HCV-796 treatment (either alone or in combination (serially or sequentially) with an additional anti-Hepatitis C virus agent).

Methods of Identifying an Individual with a Decreased Likelihood of Responding to an Anti-Hepatitis C Viral Therapy

The present invention also provides methods for identifying an individual with a decreased likelihood of responding to an anti-Hepatitis C viral therapy, comprising determining the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445 of an NS5B), and comparing the test sequence or structure with, e.g., a normal NS5B gene product sequence or structure (e.g., an NS5B sequence or structure from a reference sample). Alternatively, identifying an individual with a decreased likelihood of responding to an anti-Hepatitis C viral therapy may include determining a test sequence or structure of an NS5B gene product(s) or portion(s) thereof in a biological sample taken from a subject at a first time point, and comparing the sequence or structure of the NS5B gene product(s) or portion(s) thereof to the sequence or structure of an NS5B gene product(s) or portion(s) thereof in a biological sample taken from a subject at a second time point. A change(s) in a particular portion(s) (e.g., the HCV-796-binding pocket of an NS5B) or amino acid residue(s) of an NS5B gene product (e.g., amino acid residues 314, 316, 363, 365, 368, 414 or 445 of an NS5B) is consistent with a decreased likelihood that the individual will respond to an anti-Hepatitis C viral therapy. Closely associated methods of determining whether an individual will likely respond to an anti-Hepatitis C viral therapy with little or no resistance are also contemplated.

### Second-Generation Anti-Hepatitis C Virus Agents

The information regarding the sequence and structure of Hepatitis C RNA-dependent RNA polymerase NS5B variants that emerge in response to benzofuran (e.g., HCV-796) treatment of HCV infection is additionally useful to optimize second-generation anti-Hepatitis C agents (e.g., Hepatitis C viral inhibitors or HCV inhibitor combinations that exhibit significantly reduced, minimal, or no susceptibility to resistance caused by mutations in these variants). In addition, this information is useful in methods of selecting combinations of, e.g., anti-Hepatitis C agents and/or second-generation anti-Hepatitis C agents with additive or synergistic effects to reduce the susceptibility to resistance caused by such mutations in the Hepatitis C RNA-dependent RNA polymerase NS5B.

For example, using the HCV variants generated in response to benzofuran treatment of HCV (which may be part of a combination therapy as described herein, e.g., HCV-796 in combination with a ribavirin product and/or an interferon product), one may screen, e.g., using high throughput screening (HTS), for novel anti-Hepatitis C agents useful to treat a benzofuran treatment-resistant Hepatitis C viral infection, and thus optimize identification and chemical synthesis of second-generation anti-Hepatitis C agents. In addition, using the methods disclosed herein, one may identify a change in the amino acid sequence or structure of the benzofuran (e.g., HCV-796) binding pocket of the Hepatitis C RNA-dependent RNA polymerase NS5B generated in response to benzofuran treatment of HCV in a subject, and then administer an optimized second-generation anti-Hepatitis C agent to treat the benzofuran treatment-resistant Hepatitis C viral infection in the subject.

### Determining the Sequence or Structure of an NS5B Gene Product(S) or a Portion(s) Thereof

Determining the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445) as used in the disclosed methods may be measured in a variety of biological samples, including bodily fluids (e.g., whole blood, plasma, and urine), cells (e.g., whole cells, cell fractions, and cell extracts), and other tissues. Biological samples also include sections of tissue, such as biopsies and frozen sections taken for histological purposes. Preferred biological samples include blood, plasma, lymph, and liver tissue biopsies. It will be appreciated that analysis of a biological sample need not necessarily require removal of cells or tissue from the subject. For example, appropriately labeled agents (e.g., antibodies, nucleic acids) that interact with the HCV-796 binding pocket of an NS5B or that interact with particular amino acids (or nucleotides encoding certain amino acids) within the HCV-796 binding pocket of an NS5B, e.g., amino acid residues 314, 316, 363, 365, 368, 414 or 445, may be administered to a subject and visualized (when bound to the target) using standard imaging technology (e.g., CAT, NMR (MRI), and PET).

In diagnostic, prognostic, and monitoring assays and methods of the present invention, the sequence or structure of an NS5B gene product(s) or a portion(s) thereof (e.g., the HCV-796 binding pocket of NS5B, or particular amino acids within the HCV-796 binding pocket of NS5B, e.g., amino acid residues 314, 316, 363, 365, 368, 414 or 445) is determined to yield a test sequence or structure. The test sequence or structure is then compared with, e.g., a baseline/normal NS5B sequence or structure.

Normal sequences or structures of NS5B gene product(s) or a portion(s) thereof from different HCV genotypes, subtypes, and isolates may be determined for any particular sample type and population. Generally, baseline (e.g., normal) sequence(s) or structure(s) of an NS5B gene product(s) or a portion(s) thereof are determined by determining the sequence(s) or structure(s) of a reference NS5B gene product(s) or a portion(s) thereof from a corresponding HCV genotype and/or subtype (or isolate) that is not resistant to the anti-Hepatitis C viral therapy or anti-Hepatitis C virus agent (e.g., HCV-796) of interest. Alternatively, baseline (normal) sequence(s) or structure(s) of the NS5B gene product(s) or a portion(s) thereof may be ascertained by determining the sequence(s) or structure(s) of a reference NS5B gene product(s) or a portion(s) thereof from a sample taken from the subject prior to initiation of an anti-Hepatitis C viral therapy or administration of the anti-Hepatitis C virus agent (e.g., HCV-796) of interest.

It will be appreciated that the methods of the present invention do not necessarily require determining the entire sequence or structure of a Hepatitis C NS5B gene product(s), as determining the sequence or structure of a portion of a Hepatitis C NS5B gene product(s) is sufficient for many applications of these methods.

### Characterization of a Sequence or Structural Change in an NS5B Gene Product

The methods of the present invention involve determining the sequence or structure of a Hepatitis C RNA-dependent RNA polymerase NS5B gene product(s) or portion(s) thereof, e.g., the sequence of an NS5B polynucleotide or polypeptide (or fragment thereof, e.g., the HCV-796 binding pocket of an NS5B or the residue present at, e.g., amino acid positions 314, 316, 363, 365, 368, 414 or 445 of an NS5B). The sequence or structure of a Hepatitis C RNA-dependent RNA polymerase NS5B gene product(s) or portion(s) thereof can be measured using methods well known to those skilled in the art, those described in the Examples section (e.g., RT-PCR and crystallography), and additional techniques described herein.

One may determine changes in the amino acid sequence or structure of the HCV-796 binding pocket of the Hepatitis C RNA-dependent RNA by: (1) determining the amino acid sequence of the HCV-796 binding pocket of the Hepatitis C RNA-dependent RNA polymerase NS5B or a portion thereof; (2) determining the amino acid structure of the HCV-796 binding pocket of the Hepatitis C RNA-dependent RNA polymerase NS5B or a portion thereof; and/or (3) determining the nucleic acid sequence encoding the HCV-796 binding pocket of the Hepatitis C RNA-dependent RNA polymerase NS5B or a portion thereof.

Determination of a sequence and/or structural change(s) in an NS5B may employ various methods well known in the art, e.g., routine nucleotide sequencing (i.e., sequencing of the NS5B gene or a portion thereof (e.g., the portion(s) of the NS5B gene encoding the HCV-796 binding pocket)), PCR amplification, Northern Blotting, routine protein sequencing (i.e., sequencing of the NS5B polypeptide or a portion thereof (e.g., the portion(s) of the NS5B polypeptide responsible for interacting with HCV-796)), isoelectric focusing, spectroscopy or antibody-based detection of structural changes.

NS5B mRNA can be isolated and reverse transcribed to cDNA, and then directly sequenced by various well-known methods, or alternatively probed for the presence or absence of certain amino acid encoding sequences. Alternatively, NS5B mRNA itself may be probed for certain amino acid encoding sequences using hybridization-based assays, such as Northern hybridization, in situ hybridization, dot and slot blots, and oligonucleotide arrays. Hybridization-based assays refer to assays in which a probe nucleic acid is hybridized to a target nucleic acid. In some formats, the target, the probe, or both are immobilized. The immobilized nucleic acid may be DNA, RNA, or another oligonucleotide or polynucleotide, and may comprise naturally or normaturally occurring nucleotides, nucleotide analogs, or backbones. Methods of selecting nucleic acid probe sequences for use in the present invention (e.g., based on the nucleic acid sequence of an NS5B) are well known in the art and can be easily determined, e.g., based on the sequences set forth in SEQ ID NO: 1 and SEQ ID NO:2, which are the nucleic acid and amino acid sequences (respectively) of NS5B in wild type genotype 1b (BB7) replicon.

Alternatively, mRNA may be amplified before sequencing and/or probing. Such amplification-based techniques are well known in the art and include polymerase chain reaction (PCR), reverse-transcription-PCR (RT-PCR), PCR-enzyme-linked immunosorbent assay (PCR-ELISA), and ligase chain reaction (LCR). Primers and probes for producing and detecting amplified NS5B gene products (e.g., mRNA or cDNA) may be readily designed and produced without undue experimentation by those of skill in the art based on the nucleic acid sequences of the NS5B gene. Amplified NS5B gene products may be directly analyzed, for example, by restriction digest followed by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences.

For analysis of NS5B polypeptide structure, NS5B polynucleotides (e.g., NS5B cDNA reverse transcribed from viral RNA) may be operably linked to an expression control sequence, such as the pMT2 or pED expression vectors disclosed in Kaufman et al. (1991) Nuc. Acids Res. 19:4485-90, in order to produce NS5B polypeptides for further analysis. Many suitable expression control sequences are known in the art. General methods of expressing recombinant proteins are also known and are exemplified in Kaufman (1990) Meth. Enzym. 185:537-66. As defined herein “operably linked” means enzymatically or chemically ligated to form a covalent bond between an isolated NS5B polynucleotide and the expression control sequence in such a way that the NS5B polypeptide is expressed by a host cell that has been transformed (transfected) with the ligated polynucleotide/expression control sequence.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include other forms of expression vectors, such as viral vectors (e.g., replication defective retroviruses, adenoviruses and adeno-associated viruses), which serve equivalent functions.

The recombinant expression constructs of the invention may carry additional sequences, such as regulatory sequences (i.e., sequences that regulate either vector replication, e.g., origins of replication, transcription of the nucleic acid sequence encoding the polypeptide (or peptide) of interest, or expression of the encoded polypeptide), tag sequences such as histidine, and selectable marker genes. The term “regulatory sequence” is intended to include promoters, enhancers and any other expression control elements (e.g., polyadenylation signals, transcription splice sites) that control transcription, replication or translation. Such regulatory sequences are described, for example, in Goeddel, Gene Expression Technology: Methods in Enzymology, Academic Press, San Diego, Calif. (1990). It will be appreciated by those skilled in the art that the design of the expression vector, including the selection of regulatory sequences, will depend on various factors, including choice of the host cell and the level of protein expression desired. Preferred regulatory sequences for expression of proteins in mammalian host cells include viral elements that direct high levels of protein expression, such as promoters and/or enhancers derived from the FF-1a promoter and BGH poly A, cytomegalovirus (CMV) (e.g., the CMV promoter/enhancer), Simian virus 40 (SV40) (e.g., the SV40 promoter/enhancer), adenovirus (e.g., the adenovirus major late promoter (AdMLP)), and polyoma. Viral regulatory elements, and sequences thereof, are described in, e.g., U.S. Pat. Nos. 5,168,062; 4,510,245; and 4,968,615.

The recombinant expression vectors of the invention may carry additional sequences, such as sequences that regulate replication of the vector in host cells (e.g., origins of replication and terminator sequences) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance of the host cell transfected or transformed with the selectable marker to compounds such as G418 (geneticin), hygromycin or methotrexate. Preferred selectable marker genes include the dihydrofolate reductase (DHFR) gene (for use in dhfr⁻ host cells with methotrexate selection/amplification), the neo gene (for G418 selection), and genes conferring tetracycline and/or ampicillin resistance to bacteria.

Suitable vectors, containing appropriate regulatory sequences, including promoter sequences, terminator sequences, polyadenylation sequences, enhancer sequences, marker genes and other sequences as appropriate, may be either chosen or constructed. Inducible expression of proteins, achieved by using vectors with inducible promoter sequences, such as tetracycline-inducible vectors, e.g., pTet-On™ and pTet-Off™ (Clontech, Palo Alto, Calif.), may also be used in the disclosed methods. For further details regarding expression vectors, see, for example, Sambrook, J., E. F. Fritsch, and T. Maniatis, 1989, Molecular Cloning: A Laboratoy Manual, Cold Spring Harbor Laboratory Press, Cold Spring Harbor, N.Y. Many known techniques and protocols for manipulation of nucleic acids, for example, in preparation of nucleic acid constructs, mutagenesis, sequencing, introduction of DNA into cells, gene expression, and analysis of proteins, are also described in detail in Sambrook et al., supra.

A number of types of cells may act as suitable host cells for expression of NS5B polypeptides or polynucleotides. Suitable mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK, C3H10T½, Rat2, BaF3, 32D, FDCP-1, PC12, M1x or C2C12 cells.

Suitable bacterial cells for cloning and amplification of NS5B cDNA include various strains of E. coli, e.g., JM109, XJ Autolysis™ (Zymo Research, Orange, Calif.), BL21, and One Shot™ (Invitrogen, Carlsbad, Calif.). Common cloning vectors include pUC19, pGEX, and pBR322. Such vectors may be used for PCR amplification of cloned inserts or direct sequencing of NS5B polynucleotides.

NS5B polypeptides may also be produced by operably linking the isolated polynucleotide of the invention to suitable control sequences in one or more insect expression vectors, and employing an insect expression system. Materials and Methods for baculovirus/Sf9 expression systems are commercially available in kit form (e.g., the MAXBAC® kit, Invitrogen, Carlsbad, Calif.). Soluble forms of the polypeptides described herein may also be produced in insect cells using appropriate isolated polynucleotides as described above.

Alternatively, NS5B polypeptides may be produced in lower eukaryotes such as yeast, or in prokaryotes such as bacteria. Suitable yeast strains include Saccharomyces cerevisiae, Schizosaccharomyces pombe, Kluyveromyces strains, Candida, or any yeast strain capable of expressing heterologous proteins. Suitable bacterial strains include Escherichia coli, Bacillus subtilis, Salmonella typhimurium, or any bacterial strain capable of expressing heterologous proteins. Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment that allows correct formation of disulfide bonds (a redox system). General methods of refolding are disclosed in Kohno (1990) Meth. Enzym. 185:187-95, EP 0433225, and U.S. Pat. No. 5,399,677.

The polypeptides and polynucleotides described herein may be purified using methods known to those skilled in the art. For example, NS5B polypeptides may be concentrated using a commercially available protein concentration filter, for example, by using an AMICON® or PELLICON® ultrafiltration unit (Millipore, Billerica, Mass.). Following the concentration step, the concentrate may be applied to a purification matrix such as a gel filtration medium. Alternatively, an anion exchange resin may be employed, for example, a matrix or substrate having pendant diethylaminoethyl (DEAE) or polyethyleneimine (PEI) groups. The matrices may be acrylamide, agarose, dextran, cellulose or other types commonly employed in protein purification. Alternatively, a cation exchange step may be employed. Suitable cation exchangers include various insoluble matrices comprising sulfopropyl or carboxymethyl groups. Sulfopropyl groups are preferred (e.g., S-SEPHAROSE® columns, Sigma-Aldrich, St. Louis, Mo.). The purification of NS5B polypeptides from culture supernatant may also include one or more column steps over such affinity resins such as concanavalin A-agarose, AF-HEPARIN650, heparin-TOYOPEARL® or Cibacron blue 3GA SEPHAROSE® (Tosoh Biosciences, San Francisco, Calif.); or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify NS5B polypeptides. Affinity columns including antibodies to the protein of the invention may also be used for purification in accordance with known methods. Some or all of the foregoing purification steps, in various combinations or with other known methods, may also be employed to provide a substantially purified isolated recombinant protein. Preferably, the isolated protein is purified so that it is substantially free of other mammalian proteins.

The structure of an NS5B polypeptide (or fragments thereof) may also be determined using various well-known immunological assays employing anti-NS5B antibodies that may be generated as described herein. Immunological assays refer to assays that utilize an antibody (e.g., polyclonal, monoclonal, chimeric, humanized, scFv, and/or fragments thereof) that specifically binds to, e.g., an NS5B polypeptide (or a fragment thereof). Such well-known immunological assays suitable for the practice of the present invention include ELISA, radioimmunoassay (RIA), immunoprecipitation, immunofluorescence, fluorescence-activated cell sorting (FACS), and Western blotting. Thus, an antibody may be generated against, e.g., a portion (i.e., an epitope) of the HCV-796-binding pocket of NS5B, such that a change in a particular amino acid within the HCV-796-binding pocket may render the antibody incapable of interacting with the epitope. In this case, a negative signal (e.g., in an ELISA assay or Western Blot) indicates that an amino acid change has occurred.

An NS5B polypeptide may be used to immunize animals to obtain polyclonal and monoclonal antibodies that specifically react with the NS5B polypeptide in order to detect structural changes in a Hepatitis C RNA-dependent RNA polymerase NS5B or a portion thereof. Such antibodies may be obtained, for example, using the entire NS5B or fragments thereof as immunogens. The peptide immunogens may additionally contain a cysteine residue at the carboxyl terminus and be conjugated to a hapten such as keyhole limpet hemocyanin (KLH). Additional peptide immunogens may be generated by replacing tyrosine residues with sulfated tyrosine residues. Methods for synthesizing such peptides are known in the art, for example, as in Merrifield (1963) J. Amer. Chem. Soc. 85: 2149-54, and Krstenansky and Mao (1987) FEBS Lett. 211:10-16.

Human monoclonal antibodies (mAbs) directed against NS5B may be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., WO 91/00906, WO 91/10741, WO 92/03918, WO 92/03917, Lonberg et al. (1994) Nature 368:856-59, Green et al. (1994) Nat. Genet. 7:13-21, Morrison et al. (1994) Proc. Natl. Acad. Sci. U.S.A. 81:6851-55, and Tuaillon et al. (1993) Proc. Natl. Acad. Sci. U.S.A. 90:3720-24).

Antibodies, including monoclonal antibodies, may also be generated by other methods known to those skilled in the art of recombinant DNA technology. One exemplary method, referred to as the “combinatorial antibody display” method, has been developed to identify and isolate antibody fragments having a particular antigen specificity, and can be utilized to produce monoclonal antibodies (for descriptions of combinatorial antibody display see, e.g., Sastry et al. (1989) Proc. Natl. Acad. Sci. U.S.A. 86:5728-32; Huse et al. (1989) Science 246:1275-81; and Orlandi et al. (1989) Proc. Natl. Acad. Sci. U.S.A. 86:3833-37). After immunizing an animal with an immunogen as described above, the antibody repertoire of the resulting B cell pool is cloned. The DNA sequence of the variable regions of a diverse population of immunoglobulin molecules may be obtained using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer may be used for PCR amplification of the heavy and light chain variable regions from a number of murine antibodies (Larrick et al. (1991) BioTechniques 11: 152-56). A similar strategy may also been used to amplify human heavy and light chain variable regions from human antibodies (Larrick et al. (1991) Methods: Companion to Methods in Enzymology 2:106-10).

As used herein, the term “antibody” includes a protein comprising at least one, and typically two, VH domains or portions thereof, and/or at least one, and typically two, VL domains or portions thereof. In certain embodiments, the antibody is a tetramer of two heavy immunoglobulin chains and two light immunoglobulin chains, wherein the heavy and light immunoglobulin chains are interconnected by, e.g., disulfide bonds. The antibodies, or a portion thereof, can be obtained from any origin, including but not limited to, rodent, primate (e.g., human and nonhuman primate), camelid, shark, etc., or they can be recombinantly produced, e.g., chimeric, humanized, and/or in vitro-generated, e.g., by methods well known to those of skill in the art.

Examples of binding fragments encompassed within the term “antigen-binding fragment” of an antibody include, but are not limited to, (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; (vi) a single chain Fv (scFv; see below); (vii) a camelid or camelized heavy chain variable domain (VHH; see below); (viii) a bispecific antibody (see below); and (ix) one or more fragments of an immunoglobulin molecule fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These fragments may be obtained using conventional techniques known to those skilled in the art, and the fragments are evaluated for function in the same manner as are intact antibodies.

In some embodiments, the term “antigen-binding fragment” encompasses single domain antibodies. Single domain antibodies can include antibodies whose CDRs are part of a single domain polypeptide. Examples include, but are not limited to, heavy chain antibodies, antibodies naturally devoid of light chains, single domain antibodies derived from conventional four-chain antibodies, engineered antibodies and single domain scaffolds other than those derived from antibodies. Single domain antibodies may be any of those known in the art, or any future single domain antibodies. Single domain antibodies may be derived from any species including, but not limited to, mouse, human, camel, llama, goat, rabbit, bovine, and shark. According to at least one aspect of the invention, a single domain antibody as used herein is a naturally occurring single domain antibody known as heavy chain antibody devoid of light chains. Such single domain antibodies are disclosed in, e.g., WO 94/04678. This variable domain derived from a heavy chain antibody naturally devoid of light chain is known herein as a VHH or nanobody, to distinguish it from the conventional VH of four-chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHH molecules are within the scope of the invention.

An “antigen-binding fragment” can, optionally, further include a moiety that enhances one or more of, e.g., stability, effector cell function or complement fixation. For example, the antigen-binding fragment can further include a pegylated moiety, albumin, or a heavy and/or a light chain constant region.

Other than “bispecific” or “bifunctional” antibodies, an antibody is understood to have each of its binding sites identical. A “bispecific” or “bifunctional antibody” is an artificial hybrid antibody having two different heavy chain/light chain pairs and two different binding sites. Bispecific antibodies can be produced by a variety of methods including fusion of hybridomas or linking of Fab′ fragments; see, e.g., Songsivilai and Lachmann (1990) Clin. Exp. Immunol. 79:315-21; Kostelny et al. (1992) J. Immunol. 148:1547-53.

In addition, the present invention contemplates the use of small modular immunopharmaceutical (SMIP™) drugs (Trubion Pharmaceuticals, Seattle, Wash.). SMIPs are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Chimeric antibodies, including chimeric immunoglobulin chains, may also be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see PCT/US86/02269; EP 184,187; EP 171,496; EP 173,494; WO 86/01533; U.S. Pat. No. 4,816,567; EP 125,023; Better et al. (1988) Science 240:1041-43; Liu et al. (1987) Proc. Natl. Acad. Sci. U.S.A. 84:3439-43; Liu et al. (1987) J. Immunol. 139:3521-26; Sun et al. (1987) Proc. Natl. Acad. Sci. U.S.A. 84:214-18; Nishimura et al. (1987) Canc. Res. 47:999-1005; Wood et al. (1985) Nature 314:446-49; and Shaw et al. (1988) J. Natl. Cancer Inst. 80:1553-59).

If desired, an antibody or an immunoglobulin chain may be humanized by methods known in the art. Humanized antibodies, including humanized immunoglobulin chains, may be generated by replacing sequences of the Fv variable region that are not directly involved in antigen binding with equivalent sequences from human Fv variable regions. General methods for generating humanized antibodies are provided by Morrison (1985) Science 229:1202-07; Oi et al. (1986) BioTechniques 4:214-21; and U.S. Pat. Nos. 5,585,089, 5,693,761 and 5,693,762, all of which are hereby incorporated by reference in their entireties. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target. The recombinant DNA encoding the humanized antibody, or fragment thereof, may then be cloned into an appropriate expression vector.

Humanized or CDR-grafted antibody molecules or immunoglobulins may be produced by CDR grafting or CDR substitution, wherein one, two, or all CDRs of an immunoglobulin chain can be replaced. See e.g., U.S. Pat. No. 5,225,539; Jones et al. (1986) Nature 321:552-25; Verhoeyan et al. (1988) Science 239:1534-36; and Beidler et al. (1988) J. Immunol. 141:4053-60, all of which are hereby incorporated by reference in their entireties. U.S. Pat. No. 5,225,539 describes a CDR-grafting method that may be used to prepare humanized antibodies of the present invention (see also, GB 2188638A). All of the CDRs of a particular human antibody may be replaced with at least a portion of a nonhuman CDR, or only some of the CDRs may be replaced with nonhuman CDRs. It is only necessary to replace the number of CDRs required for binding of the humanized antibody to a predetermined antigen.

Monoclonal, chimeric and humanized antibodies, which have been modified by, e.g., deleting, adding, or substituting other portions of the antibody, e.g., the constant region, are also within the scope of the invention. For example, an antibody may be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function (e.g., altered affinity for an effector ligand, such as FcR on a cell, or the C1 component of complement) may be produced by replacing at least one amino acid residue in the constant portion of the antibody with a different residue (see, e.g., EP 388,151 A1, U.S. Pat. Nos. 5,624,821 and 5,648,260, all of which are hereby incorporated by reference in their entireties). Similar types of alterations may also be applied to murine immunoglobulins and immunoglobulins from other species. For example, it is possible to alter the affinity of an Fc region of an antibody (e.g., an IgG, such as a human IgG) for an FcR (e.g., Fc gamma R1) or for C1q binding by replacing the specified residue(s) with a residue(s) having an appropriate functionality on its side chain, or by introducing a charged functional group, such as glutamate or aspartate, or an aromatic nonpolar residue such as phenylalanine, tyrosine, tryptophan or alanine (see, e.g., U.S. Pat. No. 5,624,821).

Human antibodies to an NS5B may additionally be produced using transgenic nonhuman animals that are modified so as to produce fully human antibodies rather than the animal's endogenous antibodies in response to challenge by an antigen (see, e.g., PCT publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal that provides all the desired modifications is then obtained as progeny by crossbreeding intermediate transgenic animals containing fewer than the full complement of the modifications. One embodiment of a transgenic nonhuman animal is a mouse, and is termed the XENOMOUSE™ as disclosed in PCT publications

WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

### Methods for Decreasing the Frequency of Emergence, Decreasing the Level of Resistance, and Delaying the Emergence of a Treatment-Resistant Hepatitis C Viral Infection

The present invention provides methods for decreasing the frequency of emergence, decreasing the level of resistance, and delaying the emergence of a treatment-resistant Hepatitis C viral infection, by, e.g., administering a benzofuran inhibitor (e.g., HCV-796) of Hepatitis C virus in combination with at least one additional anti-Hepatitis C virus agent to a subject in need thereof. Benzofuran compounds and additional anti-Hepatitis C virus agents are disclosed herein. In some embodiments of the invention, the anti-Hepatitis C virus agent is an immunomodulator, particularly an interferon product, or an antiviral agent, particularly a ribavirin product.

### Pharmaceutical Compositions

In some aspects, the invention features methods for decreasing the frequency of emergence, decreasing the level of resistance, and delaying the emergence of a treatment-resistant Hepatitis C viral infection. These methods may comprise contacting a population of cells (e.g., by administering to a subject suffering from or at risk for fibrosis or a fibrosis-associated disorder) with an anti-Hepatitis C virus agent (e.g., an immunomodulator, particularly an interferon product; an antiviral agent, particularly a ribavirin product; a benzofuran, particularly HCV-796) in an amount sufficient to decrease the frequency of emergence, decrease the level of resistance, of delay the emergence of a treatment-resistant Hepatitis C viral infection.

Anti-Hepatitis C virus agents for decreasing the frequency of emergence, decreasing the level of resistance, and delaying the emergence of a treatment-resistant Hepatitis C viral infection may be used as a pharmaceutical composition when combined with a pharmaceutically acceptable carrier. Such a composition may contain, in addition to the anti-Hepatitis C virus agent(s) and carrier, various diluents, fillers, salts, buffers, stabilizers, solubilizers, and other materials well known in the art. The term “pharmaceutically acceptable” means a nontoxic or relatively nontoxic material that does not interfere with the effectiveness of the biological activity of the active ingredient(s). The characteristics of the carrier will depend on the route of administration, and are generally well known in the art.

The pharmaceutical composition of the invention may be in the form of a liposome in which an anti-Hepatitis C virus agent(s) is combined with, in addition to other pharmaceutically acceptable carriers, amphipathic agents such as lipids which exist in aggregated form as micelles, insoluble monolayers, liquid crystals, or lamellar layers which exist in aqueous solution. Suitable lipids for liposomal formulation include, without limitation, monoglycerides, diglycerides, sulfatides, lysolecithin, phospholipids, saponin, bile acids, and the like. Preparation of Such Liposomal Formulations is within the Level of Skill in the Art, as disclosed, e.g., in U.S. Pat. Nos. 4,235,871, 4,501,728, 4,837,028, and 4,737,323, all of which are incorporated herein by reference in their entireties.

As used herein, the term “therapeutically effective amount” means the amount of each active component of the pharmaceutical composition or method that is sufficient to show a meaningful subject benefit, e.g., amelioration or reduction of symptoms of, prevention of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the methods of treatment or use (including embodiments of methods for decreasing the frequency of emergence, decreasing the level of resistance, and delaying the emergence of a treatment-resistant Hepatitis C viral infection) of the present invention, a therapeutically effective amount of an anti-Hepatitis C virus agent(s) is administered to a subject, e.g., a mammal (e.g., a human). An anti-Hepatitis C virus agent(s) may be administered in accordance with the method of the invention either alone or in combination with other therapies as described in more detail herein. When coadministered with one or more agents, an anti-Hepatitis C virus agent(s) may be administered either simultaneously with the second agent, or sequentially. If administered sequentially, the attending physician will decide on the appropriate sequence of administering an anti-Hepatitis C virus agent(s) in combination with other agents.

Administration of an anti-Hepatitis C virus agent(s) used in a pharmaceutical composition of the present invention or to practice a method of the present invention may be carried out in a variety of conventional ways, such as oral ingestion, inhalation, or cutaneous, subcutaneous, or intravenous injection. Intravenous administration to the subject is sometimes preferred. When a therapeutically effective amount of an anti-Hepatitis C virus agent(s) is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil (albeit keeping in mind the frequency of peanut allergies in the population), mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the binding agent, and preferably from about 1 to 50% of the binding agent.

When a therapeutically effective amount of an anti-Hepatitis C virus agent(s) is administered by intravenous, intramuscular, cutaneous or subcutaneous injection, the binding agent will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to a binding agent, an isotonic vehicle such as sodium chloride injection, Ringer's injection, dextrose injection, dextrose and sodium chloride injection, lactated Ringer's injection, or other vehicle as known in the art. The pharmaceutical composition of the present invention may also contain stabilizers, preservatives, buffers, antioxidants, or other additive known to those of skill in the art.

The amount of an anti-Hepatitis C virus agent(s) in the pharmaceutical composition of the present invention will depend upon the nature and severity of the condition being treated, and on the nature of prior treatments that the subject has undergone. Ultimately, the attending physician will decide the amount of binding agent with which to treat each individual subject. Initially, the attending physician will administer low doses of binding agent and observe the subject's response. Larger doses of binding agent may be administered until the optimal therapeutic effect is obtained for the subject, and at that point the dosage is not generally increased further. It is contemplated that the various pharmaceutical compositions used to practice the method of the present invention should contain about 0.01 μg to about 2000 mg anti-Hepatitis C virus agent(s) per kg body weight. Dosing schedules for ribavirin products and interferon products are well known to those of skill in the art and may be found throughout the literature, e.g., in Jen et al. (2002) Clin. Pharmacol. Ther. 72:349-61, Krawitt et al. (2006) Am. J. Gastroenterol. 101:1268-73, Abonyi and Lakatos (2005) Anticancer Res. 25(2B):1315-20, Jacobson et al. (2005) Am. J. Gastroenterol. 100(11):2453-62, and Lurie et al. (2005) Clin. Gastroenterol. Hepatol. 3:610-5.

In one embodiment, pegylated interferon may be administered at a range of 0.01 μg/kg/dose to 50 μg/kg/dose, e.g., 0.1 μg/kg/dose to 3 μg/kg/dose, one or more times a week. In another embodiment, HCV-796 may be administered in doses at a range of 1 mg to 2000 mg, e.g., 50 mg to 1500 mg, one or more times a day. In another embodiment, an interferon product (including pegylated interferon), is administered intramuscularly. In yet another embodiment of the invention, ribavirin is administered orally. In yet another embodiment of the invention, HCV-796 is administered orally.

The duration of intravenous therapy using the pharmaceutical composition of the present invention will vary, depending on the severity of the disease being treated and the condition and potential idiosyncratic response of each individual subject. If administered intravenously, it is contemplated that the duration of each application of an anti-Hepatitis C virus agent(s) may be in the range of approximately 12 to 24 hours of continuous i.v. administration. Also contemplated is subcutaneous (s.c.) therapy using a pharmaceutical composition of the present invention. These therapies can be administered, e.g., daily, several times a day, weekly, biweekly, or monthly. Typically, anti-Hepatitis C viral therapy lasts from 12 to 48 weeks. It is also contemplated that where the anti-Hepatitis C virus agent is a small molecule (e.g., for oral delivery), the therapies may be administered daily, twice a day, three times a day, etc. Ultimately the attending physician will decide on the appropriate duration of i.v. or s.c. therapy, or therapy with a small molecule, and the timing of administration of the therapy using the pharmaceutical composition of the present invention.

The polynucleotide and proteins of the present invention are expected to exhibit one or more of the uses or biological activities (including those associated with assays cited herein) identified below. Uses or activities described for proteins, antibodies, or polynucleotides of the present invention may be provided by administration or use of such proteins, or antibodies, or by administration or use of polynucleotides encoding such proteins or antibodies (such as, for example, in gene therapies or vectors suitable for introduction of DNA).

### Combination Therapy

In at least one exemplary embodiment, a pharmaceutical composition comprising a benzofuran inhibitor of an NS5B (e.g., HCV-796) and at least one additional anti-Hepatitis C virus agent is administered in combination therapy. Such therapy is useful for decreasing the frequency of emergence, decreasing the level of resistance, and delaying the emergence of a treatment-resistant Hepatitis C viral infection. The term “in combination” in this context means that the benzofuran inhibitor and the at least one additional anti-Hepatitis C virus agent are given substantially contemporaneously, either simultaneously or sequentially. If given sequentially, at the onset of administration of the second compound, the first of the two compounds may still be detectable at effective concentrations at the site of treatment.

For example, the combination therapy can include at least one benzofuran inhibitor of an NS5B (e.g., HCV-796) coformulated with, and/or coadministered with, or otherwise administered in combination with, at least one additional anti-Hepatitis C virus agent. Additional anti-Hepatitis C virus agents may include at least one immunomodulator, antiviral, antifibrotics, small interfering RNA compounds, antisense compounds, polymerase inhibitors (such as nucleotide or nucleoside analogs), protease inhibitors or other small molecule anti-HCV agents, immunoglobulins, hepatoprotectants, anti-inflammatory agents, antiviral vaccine, antibiotics, anti-infectives, etc. Such combination therapies may advantageously utilize lower dosages of the administered therapeutic agents, thus avoiding possible toxicities or complications associated with the various monotherapies.

Therapeutic agents used in combination with an anti-Hepatitis C virus agent may be those agents that interfere at different stages in the autoimmune and subsequent inflammatory response. In one embodiment, at least one anti-Hepatitis C virus agent described herein may be coadministered with at least one benzofuran compound. The benzofuran compound may include any of those set forth in U.S. Provisional Patent App. Nos. 60/735,190 and 60/735,191, and U.S. Published Patent Application No. 2004/0162318.

Nonlimiting examples of the agents that can be used in combination with the benzofuran compounds described herein, include, but are not limited to, e.g., interferon products and other immunomodulators, ribavirin products, inhibitors of HCV enzymes, antifibrotics, etc. Such agents include those disclosed in Carroll et al., supra; Dhanak et al., supra; Howe et al., supra; Love et al., supra; Shim et al, supra; Summa et al., supra; Olsen et al., supra; Nguyen et al., supra; Ludmerer et al., supra; Mo et al., supra; Lu et al., supra; Leyssen et al., supra; Oguz et al., supra; U.S. Pat. No. 6,964,979; U.S. Patent Publication Nos. 2006/0063821, 2006/0040944, 2006/0035848, 2005/0159345, 2005/0075309, 2005/0059647, 2005/0049204, 2005/0048062, 2005/0031588, 2004/0266723, 2004/0209823, 2004/0077587, 2004/0067877, 2004/0028754 and 2004/0082643; and PCT Publication No. WO 2001/032153. Examples of anti-Hepatitis C virus agents include VIRAMIDINE® (Valeant Pharmaceuticals); MERIMEPODIB® (Vertex Pharmaceuticals); mycophenolic acid (Roche); amantadine; additional benzofurans; ACTILON® (Coley); BILN-2061 (Boehringer Ingelheim); Sch-6 (Schering); VX-950 (Vertex Pharmaceuticals); VALOPICITABINE® (Idenix Pharmaceuticals); JDK-003 (Akros Pharmaceuticals); HCV-796 (Wyeth/ViroPharma); ISIS-14803 (Isis Pharmaceuticals); ENBREL® (Wyeth); IP-501 (Indevus Pharmaceuticals); ID-6556 (Idun Pharmaceuticals); RITUXIMAB® (Genentech); XLT-6865 (XTL); ANA-971 (Anadys); ANA-245 (Anadys) and TARVACIN® (Peregrine).

Additional anti-Hepatitis C virus agents include immunomodulators, e.g., interferons (e.g., IFN α, β, and γ) and interferon products (e.g., pegylated interferons), which includes both natural and recombinant or modified interferons. Examples of interferon products include, but are not limited to, ALBUFERON® (Human Genome Sciences), MULTIFERON® (Viragen), PEG-ALFACON® (Inter-Mune), OMEGA INTERFERON® (Biomedicines), INTRON® A (Schering), ROFERON® A (Roche), INFERGEN® (Amgen), PEG-INTRON® (Schering), PEGASYS® (Roche), MEDUSA INTERFERON® (Flamel Technologies), REBIF® (Ares Serono), and ORAL INTERFERON ALFA® (Amarillo Biosciences).

Additional examples of anti-Hepatitis C virus agents include, but are not limited to, agents that may regulate T-cell function (e.g., thymosin alfa-1, ZADAXIN® (Sci-Clone)), agents that enhance IFN activation of immune cells (e.g., histamine dihydrochloride, CEPLEME® (Maxim Pharmaceutical)), and interferon products.

Additional anti-Hepatitis C virus agents also include antiviral agents (e.g., nucleoside analogs), such as ribavirin products, e.g., COPEGUS® (Roche); RIBASPHERE® (Three Rivers Pharmaceuticals); VIRAZOLE® (Valeant Pharmaceuticals); and REBETOL® (Schering).

### Sequence Analysis of Replicon Variants

HCV-796 has been shown to selectively inhibit HCV NS5B RNA-dependent RNA polymerase with an IC₅₀ of 40 nM in a biochemical assay. In hepatoma cells containing a subgenomic genotype 1b HCV replicon, HCV-796 reduced HCV RNA levels by 3-4 log₁₀ HCV copies/μg total RNA (EC₅₀=9 nM). Cells bearing replicon variants with reduced susceptibility to HCV-796 were generated in the presence of HCV-796 followed by G418 selection. The variant cells displayed 23- to 6812-fold resistance to HCV-796. As disclosed in greater detail in the Examples, sequence analysis of the NS5B gene derived from the replicon variants revealed several amino acid changes within 5A of the drug-binding pocket. Specifically, mutations at leucine 314, cysteine 316, isoleucine 363, serine 365 and methionine 414 of NS5B, which have been shown to directly interact with HCV-796, were observed. The impact of the amino acid substitutions on viral fitness and drug susceptibility was examined in recombinant replicons and NS5B enzymes molecularly engineered with the single amino acid mutations. The replicon variants were 10- to 200-fold less efficient in forming colonies in human hepatoma cells compared with the wild type replicon; the S365 variant failed to establish a stable cell line. Other variants (L314F, I363V, and M414V) also had 4- to 9-fold lower steady state HCV RNA levels. While different levels of resistance to HCV-796 were observed in the replicon and enzyme variants, these variants retained their susceptibility to pegylated interferon (PegIFN), ribavirin, and other HCV-specific inhibitors.

As with other RNA viruses, variants of HCV can be selected in tissue culture under drug pressure. Selection with HCV-796 using the replicon system, at concentrations 10-, 100- and 1000-times the replicon EC₅₀, resulted in variant cells that are 23-, 618- and 6812-fold, respectively, less susceptible to the compound (Table 1). Within 5 Å of the HCV-796 binding pocket, mutation of amino acids that interact with HCV-796 was observed. The frequencies of mutation are low to moderate ranging from 2% to 36% with C316Y/F/S being the most prevalent mutation (Table 2B). The resistant phenotype of the replicon variants (Tables 4 and 6) suggested these amino acids play an important role in determining the drug susceptibility to HCV-796. The replicon variants appear to be less fit than the wild type replicon based on the low colony formation efficiency (Table 5) and the reduced steady state HCV RNA levels in some variants (Table 4). At present, it is not clear whether the resistant replicon variants selected by HCV-796 can be translated into resistant viruses in vivo. If these resistant replicon variants in fact have diminished replicative fitness and are stabilized only under the selective pressure from G418, it is possible that some HCV-796-resistant virus variants that contain these mutations would not survive or would remain a minority of the HCV population in vivo. Nevertheless, selection pressure exerted by immune response in vivo is predicted to have a tremendous effect on genetic evolution of the virus. In order to assess the impact of resistance on chemotherapy, mutation frequency, population size, temporal profile and replication fitness of the resistant variants should also be considered.

As shown in Table 8, cysteine 316 in NS5B is highly conserved in HCV genotype 1a isolates. Variants at amino acid 316 in NS5B were found in genotype 1b and 4. Of 117 genotype 1b sequences reported in GenBank, 40% contains asparagine, 57% contains cysteine and 4% contains tyrosine at amino acid 316 of NS5B. Five percent (5%) of the natural isolates in genotype 4 contain asparagines at amino acid 316 of NS5B. C316Y mutation was selected in replicon-containing cells upon multiple treatments of HCV-796, the change of cysteine 316 to asparagine (C316N) has not been observed in the resistant replicons. Both tyrosine 316 and asparagine 316 replicon variants were shown to have reduced susceptibility to HCV-796. Amino acids 314, 363, 365, 368 and 414 are relatively conserved in HCV genotype 1a and 1b, which are found in 75% of the HCV-infected patients in the United States (National Institutes of Health Consensus Development Conference Statement: Management of Hepatitis C 2002 (J2002) Gastroenterology 123:2082-99) Although the resistant variants selected by HCV-796 have decreased susceptibility to HCV-796 and its related compounds, such variants remain sensitive to other anti-HCV inhibitors as well as broad-spectrum antiviral agents (Table 7). The use of these antiviral agents might help to combat the emergence of resistant viruses selected by HCV-796.

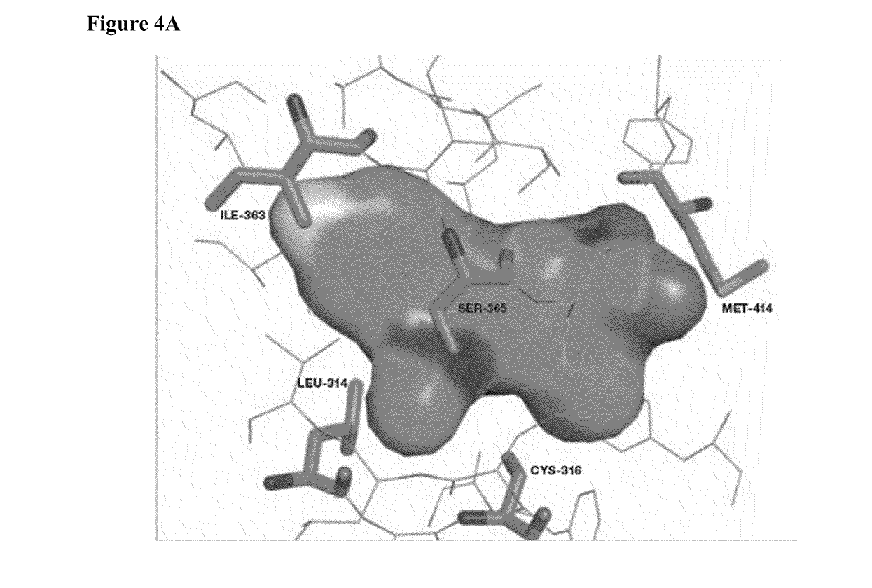

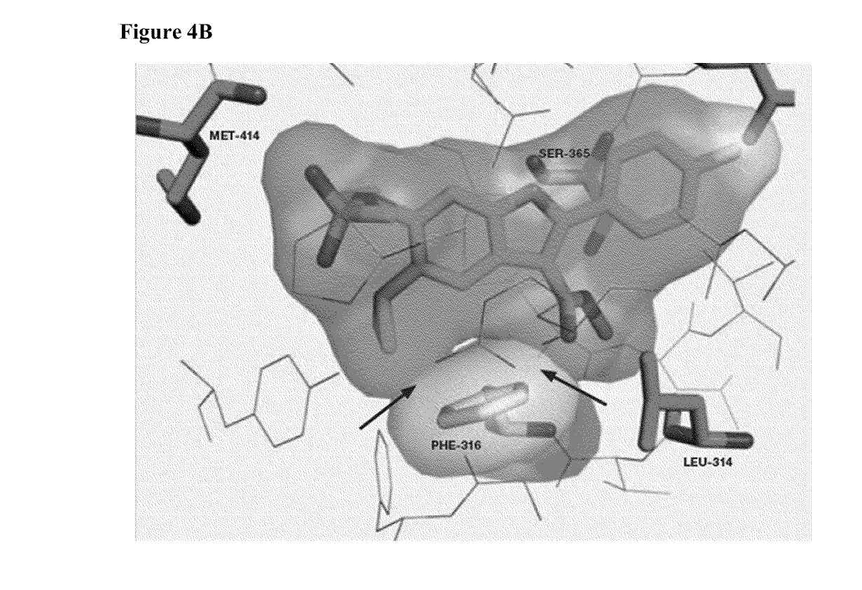

Sequence analysis of the NS5B gene derived from the 796R cells led to the identification of several amino acid changes within the NS5B protein including L314F, C316Y/F/S, I363V, S365L/A/T, S368F, and M414I/T/V. The x-ray crystal structure of HCV-796 in complex with HCV NS5B revealed that all these amino acids have direct interactions with HCV-796 (data not shown). Cysteine 316 is immediately adjacent to the catalytic triad (GDD motif; G317, D318 and D319) of the NS5B RdRp, which is reported to be important in coordinating metal ions and nucleotide triphosphate during the HCV RNA synthesis (O'Farrell et al. (2003) J. Mol. Biol. 326:1025-35). Based on the structural modeling, substitution of cysteine 316 with phenylalanine or tyrosine (C316F/Y) in NS5B resulted in strong clashes between the side chain of phenylalanine or tyrosine and both the HCV-796 and the other residues in the NS5B protein (FIG. 4). In the absence of the compound, acceptable geometry and packing can be achieved with the C316F/Y substitutions; the resulting protein conformation does not, however, permit compound binding in the observed orientation, consistent with the loss of susceptibility to HCV-796 as demonstrated in the HCV replicon (Table 4).

According to the crystal structure, NS5B protein undergoes modest conformational changes in order to accommodate the binding of HCV-796. The movement involved Arg200 and a serine-rich loop (Ser365, Cys366, Ser367, Ser368) (data not shown). Serine 365 forms a strong hydrogen bond with the amide nitrogen of HCV-796. Mutation of serine 365 to alanine (S365A) results in the loss of the hydroxyl group in serine that is the acceptor of this hydrogen bond. On the other hand, substitution of threonine for serine 365 (S365T) leads to three possibilities of rotameric configurations. In all cases, strong clashes between the side chain of threonine and the fluoro-phenyl ring or the amide group of HCV-796 were observed. The lack of hydrogen bond formation and the steric hindrance resulting from the amino acid substitutions might account for the 41- to 212-fold reduced susceptibility to HCV-796 in the S365A/T replicon variants (Table 4).

In conclusion, the inventors have verified the molecular target of HCV-796 through selection of resistant variants and mapping of amino acid changes in NS5B RdRp using the HCV replicon system. Characterization of the replicon variants identified C316Y/F/S and S365A/T as the most resistant mutations selected by HCV-796. The substitutions of amino acids at the contacting surface with HCV-796 and the resistant phenotypes suggest that the HCV replicon was under a direct antiviral pressure exerted by HCV-796, and that these amino acids play an important role in predicting the drug susceptibility to HCV-796. Although resistant to HCV-796, the replicon variants remained susceptible to pegylated interferon, ribavirin and other HCV-specific inhibitors. The use of these antiviral agents might help to combat the viral resistance selected by HCV-796. Combination of these antiviral agents might also help to reduce the emergence of resistant viruses.

The entire contents of all references, patents, and patent applications cited throughout this application are hereby incorporated by reference herein.

### EXAMPLES

The following Examples provide illustrative embodiments of the invention and do not in any way limit the invention. One of ordinary skill in the art will recognize that numerous other embodiments are encompassed within the scope of the invention.

### Example 1

### Selection of Replicon Variants with Reduced Susceptibility to HCV-796

### Example 1.1

### Materials

All tissue culture reagents were purchased from Gibco/BRL® (Invitrogen, Carlsbad, Calif.) and Hyclone (Hyclone, Logan, Utah). Clone A cells (licensed from APATH, LLC, St. Louis, Mo.) were derived from Huh-7 cells, a human hepatoma cell line. The Clone A cells contain approximately 500 to 1000 genome copies of HCV genotype 1b replicon per cell when maintained in a subconfluent monolayer in the presence of 1 mg/ml G418. The sequence of the replicon in the Clone A cells is similar to that of the genotype 1b Con 1 strain of HCV (GENBANK® accession no. AJ238799) with the exception of two mutations at NS3 (Q1112R) and NS5A (S2204I). Clone A cells were propagated in Dulbecco's minimal essential medium (DMEM; Gibco/BRL) containing 10% fetal calf serum (FCS; Hyclone) supplemented with 1% penicillin/streptomycin (GibcoBRL), 1% nonessential amino acids (Gibco/BRL), 1 mg/ml Geneticin™ (G418 sulfate; GibcoBRL) and 0.66 mM HEPES buffer, pH 7.5.

The plasmid pBB7, containing the HCV genotype 1b BB7 replicon cDNA, was also licensed from APATH, LLC. The coding sequence of pBB7 is similar to that of the genotype 1b Con 1 strain of HCV except there is one nucleotide mutation resulting in an amino acid change of S22041 within NS5A. All other molecular biology reagents were obtained from suppliers as indicated.

### Example 1.2

### Cell Culture

Approximately 3×10⁵ Clone A cells were seeded in a T-25 tissue culture flask in triplicate and cultured in medium containing 2% FCS without G418 and 0.1 or 1 μM HCV-796 dissolved in dimethyl sulfoxide (DMSO, final concentration in the medium was 0.5%, v/v). As a control, Clone A cells were passaged in parallel in the same medium containing 0.5% DMSO without compound. When the cell density reached approximately 80% confluence (about 2-3 days), the cells were split 1:3 in fresh medium containing HCV-796. An aliquot of the cells from each passage was collected to monitor the HCV RNA levels.

As the intracellular HCV viral load reduced and reached a plateau (about 16 days), fresh medium containing HCV-796 and 0.5 mg/ml G418 was added to select for cells containing the replicon variants. Approximately 20 days after the selection, small colonies of cells resistant to the inhibitor and the antibiotic became visible and were pooled. The resistant cells (796R) generated from 0.1 and 1 μM HCV-796 were named 796R (0.1 μM) and 796R (1 μM), respectively. Aliquots of 796R (0.1 μM) and 796R (1 μM) were further incubated with 10 μM HCV-796 and 0.5 mg/ml G418 to generate 796R (10 μM) cells. All resistant cells were cultured at the indicated drug concentrations in the presence of 0.5 mg/ml G418 for at least 3 weeks before analysis.

To ascertain the reproducibility of the selection, genotype 1b (BB7 isolate) replicon-containing cells were cultured in the presence of 0.1 μM or 0.2 μM of HCV-796 with 0.5 mg/ml or 1 mg/ml G418, respectively for six passages. As a control, genotype 1b (BB7 isolate) replicon-containing cells were passaged in parallel, without HCV-796.

### Example 1:3

### Results

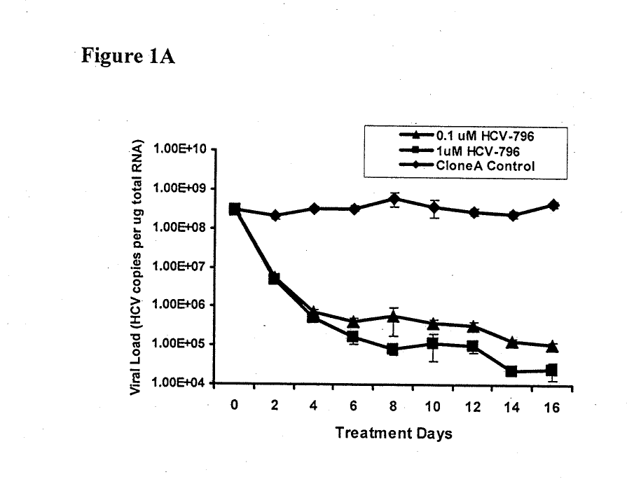

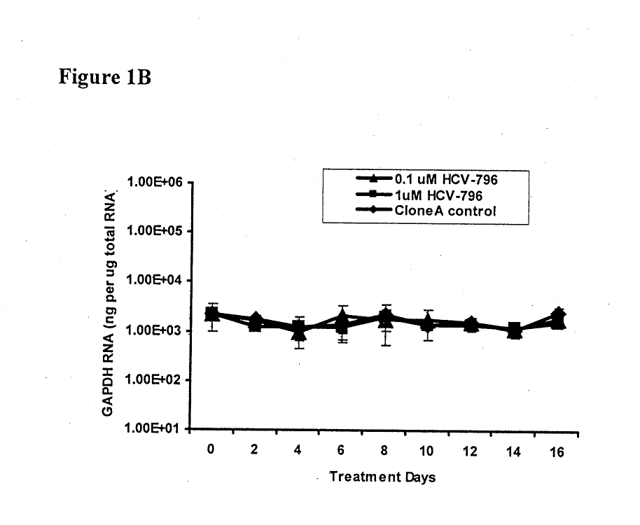

To select for HCV-796-associated replicon variants, cells bearing a genotype 1b HCV replicon were treated multiple times with 0.1 and 1 μM HCV-796 (an equivalent of 10- and 100-fold EC₅₀, respectively, for HCV-796 in a 3-day assay). At the end of the 16-day treatment, about 3.6-log₁₀ and 4.2-log₁₀ decreases in the HCV RNA levels were observed in the cells treated with 0.1 and 1 mM HCV-796, respectively (FIG. 1A). The level of a housekeeping gene, GAPDH mRNA, remained essentially unchanged throughout the 16-day period (FIG. 1B). These results suggested that HCV-796 has a direct antiviral effect on HCV replication, and that the compound is well tolerated by the cells.

The HCV replicon encodes a drug-selectable gene (neomycin phosphotransferase) that allows for selection of a functional replicon in the presence of G418. During the course of drug selection, only cells that contain replicon variants with reduced susceptibility to HCV-796 survived and gave rise to colonies. These colonies of variant cells (796R), designated as 796R (0.1 μM) and 796R (1 μM) cells, were pooled and expanded. A third pool of resistant cells [796R (10 μM)] was generated by further treating the 796R (0.1 μM) and 796R (1 μM) cells with 10 μM HCV-796.

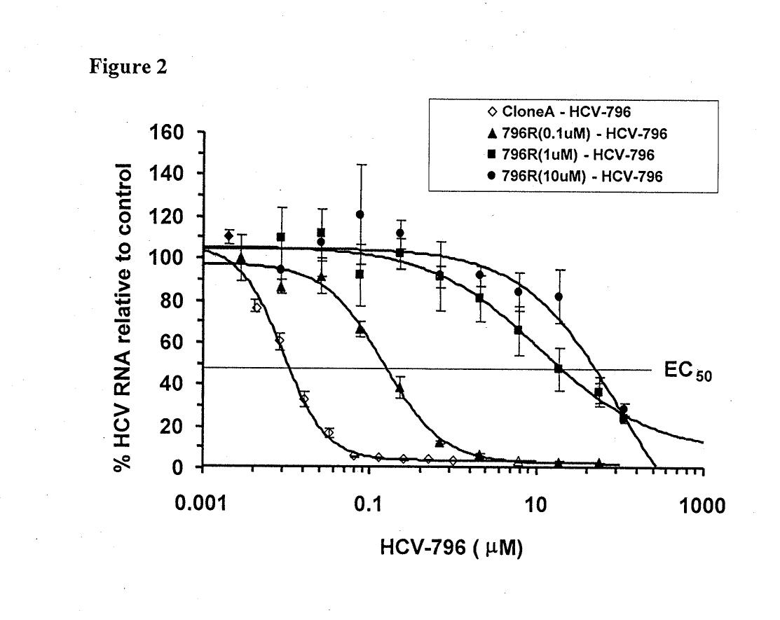

The susceptibility of the variant cells to HCV-796 was evaluated by treating the cells in the absence or presence of increasing concentrations of the compound for 72 hours. The levels of HCV RNA were determined using a quantitative TAQMAN® RT-PCR (PE Applied Biosystems, Foster City, Calif.). Incubation of the cells with HCV-796 resulted in a dose-dependent reduction of the viral RNA levels in both the control and 796R cells, suggesting that these variants were not completely resistant to the compound (FIG. 2). At the solubility limit (56 μM) of the compound in cell culture medium, HCV-796 reduced HCV RNA levels by 1.4-log₁₀, 0.7-log₁₀ and 0.5-log₁₀ in the 796R (0.1 μM), 796R (1 μM) and 796R (10 μM) cells, respectively. Control cells had a 2.1-log₁₀ reduction in the HCV RNA level (Table 1). Comparison of the EC₅₀ values for HCV-796 in the 796R cells to the control cells indicated that the replicon variants had 23- to >6812-fold reduced susceptibility to HCV-796 (Table 1). The resistant phenotype of the variant cells was confirmed in another experiment where replicon variants were selected in the presence of 0.1 and 0.2 μM HCV-796. About 25- to 65-fold reduced susceptibilities were observed among the variant cells in the second study.

### Example 2

### Mapping of Amino Acid Changes in HCV NS5B

### Example 2.1

### Isolation and Sequencing of the NS5B Gene from Replicon-Containing Cells

Total cellular RNA was extracted from the replicon-containing cells using a MICRO-TO-MIDI™ total RNA purification system (Invitrogen). The NS5B-containing cDNA was generated in a two-step RT/PCR reaction. The first strand cDNA was generated by reverse transcription (RT) in a 10 μl reaction containing 0.1 to 0.3 μg of total cellular RNA, 2 pmole of primer (7761R: 5′-CGTTCATCGGTTGGGGAGTA-3′ (SEQ ID NO:3)) and 10 nmole each of dNTPs using the SUPERSCRIPT™ first-strand synthesis system for RT-PCR (Invitrogen). The reaction was mixed, heated at 65° C. for 5 minutes and placed on ice for annealing the primer and template RNA. Ten microliters of the RNA/primer mixture were added to 9 μl of the SUPERSCRIPT™ II reaction mix, which contained 10 mM DTT, 5 μM MgCl₂ and 40 units of RNASEOUT™ RNase inhibitor (Invitrogen). After incubating the reaction mix (19 μl) at 42° C. for 2 minutes, the RT reaction was initiated by adding 1 μl of the SUPERSCRIPT™ II reverse transcriptase (50 units) (Invitrogen) followed by incubation at 42° C. for 50 minutes. The reaction was terminated at 70° C. for 15 min followed by digestion with RNase H at 37° C. for 20 min. To amplify the NS5B gene, 2 to 4 μl of the RT-reaction products were mixed with 10 pmoles each of the primers (5919F: 5′-GATCTCAGCGACGGGTCTT-3′ (SEQ ID NO:4); 7761R: as above), 10 nmoles each of dNTPs, 2 units of the Taq DNA polymerase and 1× buffer supplemented with 1.5 mM MgCl₂ provided by the supplier (Invitrogen). The reaction (final volume was 50 μL) was carried out at 95° C. for 1 min, followed by 25 cycles of (95° C. for 30 sec; 60° C. for 30 sec and 72° C. for 2 min) and an extension at 72° C. for 7 min. The PCR products were evaluated by agarose gel electrophoresis. The band at 1.8 kb was excised, and the cDNA fragment was extracted from the gel. The cDNA was ligated with the PCR4-TOPO™ vector (Invitrogen), and the resulting recombinant DNA plasmid was transformed into the ONE SHOT® chemical-competent E. coli according to manufacturer's instruction (TOPO® TA CLONING kit for sequencing (Invitrogen)). The presence of the HCV NS5B insert in the plasmids was verified by EcoRI digestion. Plasmids containing the HCV NS5B inserts were subjected to nucleotide sequencing using ABI PRISM® BIGDYE® terminator cycle sequencing ready reaction kit v3.0 (Applied Biosystems, Foster City, Calif.). The sequencing reactions were set up in a 96-well PCR plate in a final volume of 20 μl. The reaction mix consisted of 1 μl of the terminator-ready reaction mix, 3.5 μl of 5× sequencing buffer, 3.2 pmoles of primer and 500 ng of plasmid DNA. The sequence reaction was conducted under the conditions as per the manufacturer's instruction. The sequenced products were gel purified using DYEEX™ 96 Kit (Qiagen, Valencia, Calif.), dried down, denatured with formaldehyde, and separated by electrophoresis using an ABI PRISM® 3700 DNA Sequencer (Applied Biosystems). Sequence data were analyzed using SEQUENCHER® v4.0 (Gene Codes Corp., Ann Arbor, Mich.).

### Example 2.2

### Results

HCV-796 is a potent and selective inhibitor that inhibits the HCV NS5B RdRp (data not shown). Crystal structure of the NS5B in complex with HCV-796 showed that HCV-796 binds near the catalytic site in the palm domain of the enzyme (data not shown). Therefore, it is likely that the resistance observed in the 796R cells was due to mutations within NS5B. To map the amino acid changes within the NS5B, total cellular RNA was extracted from the 796R cells. The gene segment encoding the NS5B was amplified by RT-PCR followed by cloning and transforming into E. coli. Ninety-three bacterial clones containing a full-length NS5B gene were sequenced. In addition, eleven clones containing the NS5B gene derived from the control Clone A cells were used as comparators.

As shown in Table 2A, the NS5B prepared from the control cells contained random amino acid changes with no specific patterns. A total of 32 amino acid changes among the 11 clones were observed, with an average of 3 amino acid changes per clone. All amino acid changes contain one nucleotide change per amino acid resulting in a mutation rate of 1.6×10⁻³ mutations per nucleotide for the HCV replicon.

Several unique mutations within the NS5B, which were not found in the control cells, were observed in the 93 clones derived from the 796R cells (Table 2B). Of particular interest are the mutations within 5 Å of the HCV-796 binding pocket, which include: amino acid 316 (Cys to Tyr, 10 clones; Cys to Phe, 17 clones; Cys to Ser, 6 clones), 363 (Ile to Val, 4 clones), 365 (Ser to Leu, 23 clones; Ser to Ala, 3 clones; Ser to Thr, 4 clones), 368 (Ser to Phe, 2 clones) and 414 (Met to Ile, 11 clones; Met to Thr, 2 clones). An additional change at amino acid 314 (Leu to Phe) was observed in the second study. As illustrated in FIG. 3A, the key amino acid substitutions are distributed among five structural components within the drug-binding pocket; namely, the active site loop, the serine-rich (Cys³⁶⁶) loop, and the α-helix M, α-helix G and Tyr⁴⁴⁸ loop. Amino acids L314 and C316 are within the active site loop, I363, S365 and S368 are in the serine-rich loop, and M414 mutation is in the α-helix M. All these amino acids have direct interactions with HCV-796 as identified in the crystal structure of the NS5B-HCV-796 complex (FIG. 3B). Most of the mutations occurred with a frequency in the range from 2-18%, with the exception of C316Y/F/S, S365L/T/A and C445F, which occurred in 36%, 31% and 54%, respectively (Table 2B). Cysteine 445 is located proximally to the HCV-796 binding pocket. The substitution of C445F was frequently found in replicon variants selected from other classes of HCV polymerase inhibitors.

To assess if there is any pattern of mutations within NS5B in the replicon variants, amino acid substitutions that only appeared in combination with other substitutions were evaluated. Amino acid substitutions that were found in the DMSO-treated control cells, and occurred only once were considered random mutations, and not included in the evaluation. Using these criteria, a total of 24 amino acid changes within the NS5B were observed (Table 2B). Close examination of the amino acid changes revealed seven patterns of mutations (Table 3). K355R and C445F were found in all three pools of 796R cells. V85L, F162Y and C316F, with or without T19P; and C316S/Y and C445F were found in replicon variants selected from 1 and 10 μM HCV-796. The remaining three combinations: P197A, C445F and V581A; C316Y and M414I; and S365L and T390I were found in either 796R(1 μM) or 796R(10 μM) variant cells. In some replicon variants, C445F or S365L existed as the sole amino acid change (Table 2B).

### Example 3

### Characterization of the Amino Acid Substitutions in Replicon Variants

### Example 3.1

### Construction of the BB7 Replicon Variant Plasmids

Standard recombinant DNA technology was used to construct and purify BB7 replicon variant plasmids. All NS5B variants were initially generated using the plasmid NS5B-BB7dCT21-His as the input template (Howe et al. (2004) Antimicrobial Agents Chem. 48:4813-21). Single nucleotide changes were introduced using the QUIKCHANGE® XL Site Directed Mutagenesis kit (Stratagene, La Jolla, Calif.) according to the manufacturer's procedure. The sequences of the oligonucleotide primers used for the site directed mutagenesis are indicated as follows (F (forward) and R (reverse)):

L314F(c940t-F)

(SEQ ID NO: 5)

5′-AGGACTGCACGATGTTCGTATGCGGAGACG-3′
L314F(c940t-R)

(SEQ ID NO: 6)

5′-CGTCTCCGCATACGAACATCGTGCAGTCCT-3′
C316F(g947t-F)

(SEQ ID NO: 7)

5′-GCACGATGCTCGTATTCGGAGACGACCTTGTC-3′
C316F(g947t-R)

(SEQ ID NO: 8)

5′-GACAAGGTCGTCTCCGAATACGAGCATCGTGC-3′
C316S(t946a-F)

(SEQ ID NO: 9)

5′-GCACGATGCTCGTAAGCGGAGACGACCTTG-3′
C316S(t946a-R)

(SEQ ID NO: 10)

5′-CAAGGTCGTCTCCGCTTACGAGCATCGTGC-3′
S365L(c1094t-F)

(SEQ ID NO: 11)

5′-GACTTGGAGTTGATAACATTATGCTCCTCCAATGTGTCAG-3′
S365L(c1094t-R)

(SEQ ID NO: 12)

5′-CTGACACATTGGAGGAGCATAATGTTATCAACTCCAAGTC-3′
S365A(t1093g-F)

(SEQ ID NO: 13)

5′-CTTGGAGTTGATAACAGCATGCTCCTCCAATGTG-3′
S365A(t1093g-R)

(SEQ ID NO: 14)

5′-CACATTGGAGGAGCATGCTGTTATCAACTCCAAG-3′
S365T(t1093a-F)

(SEQ ID NO: 15)

5′-GACTTGGAGTTGATAACAACATGCTCCTCCAATGTGTC-3′
S365T(t1093a-R)

(SEQ ID NO: 16)

5′-GACACATTG GAGGAG CAT GTTGTTATCAACTCCAAGTC-3′
S368F(c7085t-F)

(SEQ ID NO: 17)

5′-GATAACATCATGCTCCTTCAATGTGTCAGTCGCG-3′
S368F(c7085t-R)

(SEQ ID NO: 18)

5′-CGCGACTGACACATTGAAGGAGCATGATGTTATC-3′
M414T(t1241c-F)

(SEQ ID NO: 19)

5′-TAGGCAACATCATCACGTATGCGCCCACCTTG-3′
M414T(t1241c-R)

(SEQ ID NO: 20)

5′-CAAGGTGGGCGCATACGTGATGATGTTGCCTA-3′
M414V(a1240g-F)

(SEQ ID NO: 21)

5′-CTAGGCAACATCATCGTGTATGCGCCCACCTT-3′
M414V(a1240g-R)

(SEQ ID NO: 22)

5′-AAGGTGGGCGCATACACGATGATGTTGCCTAG-3′

To prepare expression plasmid NS5B-BB7dCT21-His(C316Y), a point mutation was made in plasmid NS5B-BB7dCT21-His to change the TGC codon (cysteine) to a TAC codon (tyrosine). To prepare expression plasmid NS5B-BB7dCT21-His(C316N), a double point mutation was made in plasmid pRSET-BB7dCT21-His to change the TGC codon (cysteine) to an AAC (asparagine). To prepare expression plasmid NS5B-BKdCT21(N316C), a double point mutation was made in plasmid pRSET-BKdCT21-His to change the AAC (asparagine) to a TGC codon (cysteine). To prepare expression plasmid NS5B-BB7dCT21-His(M414I), a point mutation was made in plasmid NS5B-BB7dCT21-His to change the ATG codon (methionine) to an ATC codon (isoleucine). To prepare expression plasmid NS5B-BB7dCT21-His(I363V), a point mutation was made in plasmid NS5B-BB7dCT21-His to change the ATA codon (isoleucine) to a GTA codon (valine). Individual clones were sequenced to confirm for the presence of the desired mutations and lack of other changes.

To prepare pBB7-L314F, pBB7-C316F/S/Y/N, pBB7-I363V, pBB7-S365L/A/T, pBB7-S368F and pBB7-M414/T/V/I the Bsu36I fragments from plasmids NS5B-BB7dCT21-His(L314F), NS5B-BB7dCT21-His(C316F/S/Y/N), NS5B-BB7dCT21-His(I363V), NS5B-BB7dCT21-His(S365L/A/T), NS5B-BB7dCT21-His(S368F) and NS5B-BB7dCT21-His(M414T/V/I), were cloned into the pHCVrep1b.BB7 (licensed from APATH LLC) backbones digested with Bsu36I. The pBB7-plasmids were sequenced to confirm the expected single nucleotide changes in the coding sequence for NS5B.

### Example 3.2

### RNA Transcription and Electroporation of Cultured Cells

pBB7-replicon variant DNAs were linearized with Sca I, and in vitro transcription was performed using Ambion's MEGASCRIPT® T7 High Yield Transcription kit (Austin, Tex.). Purified RNA transcripts were electroporated into Huh-7 cells in quadruplicates using a Biorad GENE PULSER® Electroporation System (Setting: 270V, 950 μF) (Hercules, Calif.). Stably transfected replicon variant cell lines were initially selected with 0.25 mg/ml G418 and stepped up to 1 mg/ml before further testing. One cell plate was stained with Crystal Violet to visualize the number of colonies and determine the colony formation efficiency. Individual cell clones from each plate were pooled and expanded for drug susceptibility testing. The NS5B gene of each replicon variant at an early passage was sequenced to confirm the presence of the expected nucleotide changes in the coding region for NS5B. No other changes affecting the amino acid sequence of NS5B were observed.

### Example 3.3

### Expression and Purification of NS5B Enzyme Variants

All NS5B enzymes were expressed and purified according to the protocol for NS5B-BB7dCT21-His as described (Howe et al. (2004) Antimicrobial Agents Chem. 48:4813-21). Briefly, expression plasmids were transformed into E. coli cells and NS5B expression was initiated by the addition of isopropyl-beta-D-thiogalactopyranoside (IPTG). After 4 to 6 hours of incubation the cells were harvested and lysed. NS5B enzymes were purified by chromatography using a nickel affinity column (Talon, BD Biosciences, Clontech Laboratories, Inc., Mountain View, Calif.)) followed by a cation exchange column (Poros HS, Applied Biosystems, Foster City, Calif.).

### Example 3.4

### Results

The contribution of individual amino acid changes on drug resistance was assessed in replicon variants containing single amino acid mutations in NS5B in the background of the genotype 1b, BB7 adaptive replicon (Blight et al. (2000) Science 290:1972-74). The replicon variants were tested in the absence or presence of elevating concentrations of HCV-796 in a 3-day assay. Within the active site loop, the change of amino acid 314 from leucine to phenylalanine (L314F) did not change the susceptibility to HCV-796 (Table 4) in the replicon. In contrast, the substitutions of cysteine 316 with phenylalanine or tyrosine or serine (C316F/Y/S) resulted in EC₅₀ values of 392, 501 and 30 nM, which were 130-, 166- and 10-fold, respectively, greater than that of the wild type 1b, BB7 replicon (Table 4). Another replicon variant, C316N, which was not found in the replicon resistance selection, but was reported to make up 40% of the NS5B sequences of natural isolates in the NIH genetic sequence database (GenBank), displayed over 26-fold reduced susceptibility to HCV-796.

While changes in residues 363 (I363V) and 368 (S368F) within the serine-rich loop had a modest effect on the susceptibility to HCV-796, substitutions of serine 365 with alanine or threonine (S365A/T) led to 41- and 212-fold reduced susceptibility to the compound, respectively (Table 4).

In the α-helix M, the substitutions of methionine 414 with isoleucine or valine (M414I/V) resulted in low to moderate increases in replicon EC₅₀ values leading to 3-8 fold reduced susceptibility to HCV-796 (Table 4). The change of methionine 414 to threonine did not change the susceptibility to HCV-796 in the replicon.

The impact of amino acid substitutions on viral fitness and growth kinetics was estimated based on colony formation efficiency and steady-state HCV RNA levels in the replicon-containing cells. Transfection of the replicon RNAs into Huh-7 cells resulted in colony formation in the presence of G418 within 20 days after transfection. No colonies were obtained from Huh-7 cells transfected with the RNAs containing a GAA mutation within the NS5B or mock transfected (result not shown). As shown in Table 5, the colony formation efficiencies for the replicon variants were on the order of 10- to 200-fold less than that of the wild type BB7 replicon, suggesting that the amino acid substitution in NS5B might have an adverse effect on viral fitness. The steady-state HCV RNA levels in the replicon variants L314F, I363V and M414V were 4- to 9-fold less as compared to the wild type BB7 replicon, and for S365L it failed to generate a stable cell line (Table 4). It is likely that the mutations within NS5B in these replicon variants have introduced a deleterious effect to the viral replication. It should be noted that comparable steady state levels of HCV RNA were observed in the pools of 796R and control Clone A cells (Table 1). It is possible that compensatory mutations might have occurred in other parts of the replicon genome hence restoring the viral RNA to the wild type levels.

### Example 4

### Inhibitory Activity of HCV-796 in Mutant NS5B Enzymes

To assess the effect of HCV-796 on polymerase activity in the replicon variants, recombinant genotype 1b, BB7 NS5B enzymes molecularly engineered with single substitutions at amino acids 316, 414 and 363 were cloned and expressed in E. coli. The polymerase activity of the purified mutant enzymes was evaluated in a biochemical assay in the absence or presence of increasing concentrations of HCV-796. Similar to the replicon variants, the polymerase variants displayed a reduced susceptibility to HCV-796 as compared to the wild type enzyme, although the levels of resistance were substantially attenuated. Among the enzyme variants, the substitutions of amino acid 316 from cysteine to asparagine or tyrosine or phenylalanine (C316N/Y/F) resulted in 2- to 125-fold reduced susceptibility to HCV-796, whereas the substitutions of methionine 414 to valine or isoleucine (M414V/I), and the substitution of isoleucine 363 to valine (I363V) showed no appreciable difference in drug susceptibility to the compound (Table 6).

In the biochemical assay, the recombinant HCV NS5B enzymes from the genotype 1b isolates BK and J4, which each contain an asparagine at position 316, are less susceptible to HCV-796 than those that contain a cysteine at this position (data not shown). To ascertain if asparagine and cysteine have opposite effects on the susceptibility to HCV-796, the NS5B enzyme derived from the genotype 1b BK isolate was engineered with a single asparagine to cysteine change at amino acid 316 (BK-N316C). This enzyme variant was 4.5-fold more susceptible to HCV-796 than the wild type BK enzyme (Table 6) confirming the importance of this residue on drug susceptibility to HCV-796.

### Example 5

### Activities of Antiviral Agents in HCV-796-resistant Replicon-Containing Cells

### Example 5.1

### Evaluation of Antiviral Agents in Replicon Variants

Drug susceptibility of the replicon-containing cells to various compounds was evaluated as described previously (Howe et al. (2004) Antimicrobial Agents Chem. 48:4813-21). Briefly, cells were treated with increasing concentrations of compounds in medium containing 2% FCS and no G418 for three days at 37° C. and 5% CO₂. After incubation, total RNA from the replicon-containing cells was isolated. The levels of HCV, glyceraldehyde 3-phosphate dehydrogenase (GAPDH) and ribosomal (rRNA) RNAs were quantified using TAQMAN® (PE Applied Biosystems, Foster City, Calif.) reverse transcriptase PCR reactions. The amounts of HCV, 18S ribosomal, and GAPDH RNAs in each sample were estimated by comparing the number of cycles during the exponential phase of the PCR amplification with those in the corresponding standard curves. HCV RNA standards used for the construction of the standard curve were prepared by extracting the total RNA from the Clone A cells. The RNA sample was sent to National Genetics Institute to quantify HCV RNA. Total RNA extracted from Clone A cells was quantified by O.D.₂₆₀ measurement and used for construction of the standard curves of rRNA and GAPDH. The concentrations of the compounds that inhibit 50% of the HCV RNA level (EC₅₀) were determined using the MDL® LIFE SCIENCE WORKBENCH® (LSW) Data Analysis software (MDL Information Systems, San Leandro, Calif.) in Microsoft EXCEL®. The amounts of HCV or GAPDH RNAs in the samples were expressed as HCV RNA (copies) or GAPDH (ng), respectively, per μg of total RNA using rRNA as a marker for total RNA measurement.

### Example 5.2

### Results

The antiviral activities of panel of antiviral agents, including two broad-spectrum antiviral agents and an HCV-specific inhibitor, were evaluated in the C316Y replicon variant and pools of variant cells selected from HCV-796. Pegylated interferon and ribavirin, both of which have demonstrated antiviral activities against many viruses (Akahane et al. (1999) J. Med Virol. 58:196-200; Hartman et al. (2003) Ped. Infect. Disease J. 22:224-9; Lanford et al. (2003) J. Virol. 77:1092-104; McCormick et al. (1984) Lancet 2:1367-9; McCormick et al. (1986) N. Engl. J. Med. 314:20-6; Umemura et al. (2002) Hepatology 35:953-9; Yu et al. (2001) Antiviral Res. 52:241-9), inhibit HCV replication in C316Y replicon variant as efficiently as in the wild type replicon (Table 7). Ribavirin also inhibits replicon variants containing other HCV-796-associated amino acid mutations (data not shown).

The activity of the pyranoindole HCV polymerase inhibitor HCV-371 ([(1R)-5-cyano-8-methyl-1-propyl-1,3,4,9-tetrahydropyano[3,4-b]indol-1-yl]acetic acid) was also evaluated against the replicon variants. HCV-371 has been shown to bind at a different site in NS5B than that for HCV-796 (Howe et al. (2004) Antimicrobial Agents Chem. 48:4813-21). In contrast to HCV-796, HCV-371 inhibited both the wild type and C316Y replicons with similar activities (Table 7).

Taken together, these results suggest that the resistance selected by HCV-796 is specific to the benzofuran class of inhibitors, and that the replicon variants remain sensitive to pegylated interferon, ribavirin and other anti-HCV compounds.

TABLE 1
Activity of HCV-796 Against Replicon Variants

| | | | Fold | Viral Load | |
|---|---|---|---|---|---|
| | | | Reduced | (copies/μg total | Mean log₁₀ |
| Cells^{a} | EC₅₀ (μM) ± SD^{b} | | Susceptibility | RNA) ± SD | Viral Reduction^{c} |
| CloneA | 0.013 ± 0.013 | (n = 8) | — | 3.7 ± 2.4 × 10⁸ | 2.1 ± 0.4 (0.7 μM) |
| 796R (0.1 μM) | 0.3 ± 0.2 | (n = 4) | 23 | 2.9 ± 0.5 × 10⁸ | 1.4 ± 0.3 (56 μM) |
| 796R (1 μM) | 8.0 ± 5.2 | (n = 12) | 618 | 2.4 ± 2.0 × 10⁸ | 0.7 ± 0.2 (56 μM) |
| 796R (10 μM) | >88.0 | (n = 4) | >6812 | 4.1 ± 1.7 × 10⁸ | 0.5 ± 0.3 (56 μM) |

^{a}796R represents cells that are less susceptible to HCV-796. Concentrations of HCV-796 used for the selection are indicated in parentheses.
^{b}EC₅₀ values were determined using the MDL LSW data analysis ™. Inhibitory activity is expressed as mean EC₅₀ ± standard deviation. n indicates number of determinations.
^{c}Viral load reduction was determined at the indicated compound concentrations in parenthesis in a 3-day assay. Data represent the mean log reduction of viral RNA ± standard deviation. Results represent at least 3 independent determinations.

TABLE 2A
Amino Acid Changes in NS5B Derived from Clone A Control Cells

| | amino acid | | | | | | | | | | | | | | |
|---|---|---|---|---|---|---|---|---|---|---|---|---|---|---|---|
| | 5 | 53 | 57 | 79 | 114 | 116 | 117 | 122 | 125 | 140 | 182 | 231 | 253 | 322 | 345 |
| pBB7 | T | T | L | K | K | V | N | V | D | A | L | N | I | V | R |
| p7-1 | | | | | | I | | | | | | | | | G |
| p7-2 | A | | | | | | | | N | | P | | | | |
| p7-3 | | A | | | R | | S | | | | | | | | |
| p7-4 | | | | R | | | | | | | | D | | | |
| p7-5 | | | | R | | | | | | | | D | | | |
| p7-6 | | | | | | | | | | | | | | | |
| p7-7 | | | | | | | | A | | | | | T | A | |
| p7-8 | | | | | | | | | | V | | | | | |
| p7-9 | | | | | | | | | | | | | | | |
| p7-10 | | | P | | | | | | | | | | | | |
| p7-11 | | | | | | | | | | | | | | | |

| | amino acid | | | | | | | | | | | | |
|---|---|---|---|---|---|---|---|---|---|---|---|---|---|
| | 353 | 369 | 407 | 412 | 424 | 432 | 443 | 465 | 471 | 478 | 531 | 533 | 556 |
| pBB7 | P | N | S | I | I | I | L | R | A | S | R | K | S |
| p7-1 | | | P | | | V | | | | | | E | |
| p7-2 | | | | | | | | | T | | G | | |
| p7-3 | | | | T | | | | | | | | | |
| p7-4 | | S | | | T | | | | | | | | |
| p7-5 | | S | | | T | | | | | | | | |
| p7-6 | | | | | V | V | | | | P | | | |
| p7-7 | | | | | | | | | | | | | |
| p7-8 | | | | | | | | Q | | | | | |
| p7-9 | L | | | | | | | | | | | | G |
| p7-10 | | | | | | | | | | | | | |
| p7-11 | | | | | | | P | | | | | | |

TABLE 2B
Amino Acid Changes in NS5B Derived from 796R Cells

| | | amino acid | | | | | | | | | | | | |
|---|---|---|---|---|---|---|---|---|---|---|---|---|---|---|
| Clones | | 4 | 19 | 85 | 97 | 147 | 162 | 181 | 197 | 201 | 286 | 316 | 329 | 355 |
| isolated | pBB7 | Y | T | V | A | V | F | T | P | V | T | C | T | K |
| 0.1 μM | p1-4 | | | | | | | | | | | | | R |
| HCV-796 | p1-10 | | | | | | | | | | | | | R |
| | p1-26 | | | | | | | | | | | | | R |
| | p1-27 | | | | | | | | | | | | | R |
| | p1-25 | | | | | | | | | | | | | R |
| | p3-8 | | | | | | | | | | | | | |
| | p3-22 | | | | | | | | | | | | | |
| | p3-23 | | | | | | | | | | | | | |
| | p3-2 | | | | | | | | | | | | | |
| | p3-11 | | | | | | | | | | | | | |
| | p3-5 | | | | | | | | | A | | | | |
| | p3-14 | | | | | | | | | A | | | | |
| | p1-5 | | | | | | | | | | | | | |
| | p1-28 | | | | | | | | | | | | | |
| | p3-1 | | | | | | | | | | | | | |
| | p1-1 | | | | | | | | | | | | | |
| | p1-2 | | | | | | | | | | | | | |
| | p1-3 | | | | | | | | | | | | | |
| | p3-4 | | | | | | | | | | | | | |
| | p3-28 | | | | | | | | | | | | | |
| 1 μM | p4-2 | | P | L | | | Y | | | | | F | | |
| HCV-796 | p4-3 | | P | L | | | Y | | | | | F | | |
| | p4-4 | | P | L | | | Y | | | | | F | | |
| | p4-5 | | | L | | | Y | | | | | F | | |
| | p4-7 | | P | L | | | Y | | | | | F | | |
| | p4-8 | | | L | | | Y | | | | | F | | |
| | p4-10 | | | L | | | Y | | | | | F | | |
| | PCR4 | | | L | | | Y | | | | | F | | |
| | PCR4M | | | L | | | Y | | | | | F | | |
| | p4-9 | | | L | | | Y | | | | | | | |
| | p6-10 | | | | | | | | | | | | | |
| | PCR6 | | | | | | | | A/P | | | | | |
| | p9-8 | | | | | | | | | | | | | |
| | p9-9 | | | | | | | | | | | | | |
| | p6-1 | | | | | | | | A | | | | | |
| | p6-2 | | | | | | | | A | | | | | |
| | p6-3 | | | | | | | | A | | | | | |
| | p6-11 | | | | | | | | A | | | | | |
| | p6-26 | | | | | | | | A | | | | | |
| | p6-8 | | | | | | | | A | A | | | | |
| | p6-12 | | | | | | | | A | | | | | |
| | p9-2 | | | | | | | | | A | | | | |
| | p9-13 | | | | | | | | | A | | S | | |
| | p9-1 | | P | | | | | | | | | S | | |
| | p9-3 | | | | | | | | | | | S | | |
| | p9-4 | | P | | | | | | | | | S | | |
| | p9-7 | | P | | | | | | | | | S | | |
| | PCR9 | | | | | | | | | | | S/C | | |
| | p9-5 | | | | | | | | | | | | | R |
| | p9-6 | | | | | | | | | | | | | R |
| | p4-14 | | | | | | | | | | | | | |
| | p6-4 | | | | | | | | | | | | | |
| | p6-7 | | | | | | | | | | | | | |
| | p9-14 | | | | | | | | | | | | | |
| 10 μM | p41-1 | | | | | | | | | | | Y | | |
| HCV-796 | p41-2 | | | | | | | | | | | Y | | |
| | p41-3 | | | | | | | | | | | Y | | |
| | p41-5 | | | | | | | | | | | Y | | |
| | p41-15 | | | | | | | | | | | Y | | |
| | PCR41 | | | | | | | | | | | C/Y | | |
| | p41-7 | | | | | | | | | | | Y | | |
| | p41-17 | | | | | | | | | | | | | |
| | p49-3 | | | | | | | | | | | Y | | R |
| | p41-4 | | | | | | | | | | | Y | | |
| | p41-6 | | A | | | | | | | | | Y | | |
| | p44-14 | | P | L | | | Y | | | | | F | | |
| | p44-15 | | P | L | | | Y | | | | | F | | |
| | p44-20 | | P | L | | | Y | | | | | F | | |
| | p44-32 | | P | L | | | Y | | | | | F | | |
| | p44-16 | | P | L | L | | Y | | | | | F | | |
| | p44-23 | | | L | | | Y | | | | | F | | |
| | p44-13 | | | L | | | Y | | | | | F | | |
| | PCR44 | | | L | | | Y/F | | | | | C/F | | |
| | p46-15 | H | | | | I | | I | | | | | I | |
| | p46-16 | H | | | | I | | I | | | | | I | |
| | p49-5 | | | | | | | | | | | | I | R |
| | PCR46 | | | | | | | | | | | | | R |
| | p46-7 | | | | | | | I | | | | | | |
| | p46-14 | | | | | | | I | | | | | | |
| | p46-8 | | | | | | | | | | | | | |
| | p46-12 | | | | | | | | | | A | | | |
| | p46-9 | | | | | | | | | | M | | | |
| | p46-11 | | | | | | | | | | | | | |
| | p46-13 | | | | | | | | | | | | | |
| | p44-18 | | | | | | | | | | | | | |
| | p41-13 | | | | V | | | | | | | | | R |
| | p49-1 | | | | | | | | | | | | | |
| | p49-4 | | | | | | | | | | | | | |
| | p49-2 | | A | | | | | | | | | | | R |
| | PCR49 | | | | | | | | | | | | | R |
| | p44-12 | | | | | | | | | | | | | |
| | p44-22 | | | | | | | | | | | | | |
| | p46-10 | | | | | | | | | | | | | |

| | | | | | | | | | | | | | |
|---|---|---|---|---|---|---|---|---|---|---|---|---|---|
| No. of clones out of | 2 | 14 | 18 | 2 | 2 | 18 | 4 | 8 | 5 | 3 | 33 | 3 | 13 |
| 93 clones | | | | | | | | | | | | | |
| Frequency of amino | 2.2 | 15.1 | 19.4 | 2.2 | 2.2 | 19.4 | 4.3 | 8.6 | 5.4 | 3.2 | 35.1 | 3.2 | 14.0 |
| acid substitution | | | | | | | | | | | | | |

| | | amino acid | | | | | | | | | | |
|---|---|---|---|---|---|---|---|---|---|---|---|---|
| Clones | | 363 | 365 | 368 | 390 | 414 | 440 | 442 | 445 | 514 | 534 | 581 |
| isolated | pBB7 | I | S | S | T | M | E | A | C | Q | L | V |
| 0.1 μM | p1-4 | | | | | | | | F | | F | |
| HCV-796 | p1-10 | | A | | | | | | F | | F | |
| | p1-26 | | | | | T | | | F | | V | |
| | p1-27 | | | | | | | | F | | V | |
| | p1-25 | | | | | | | | F | | F | |
| | p3-8 | | | | | I | | | F | | | |
| | p3-22 | | | | | I | | | F | | | |
| | p3-23 | | | | | I | | | F | | | |
| | p3-2 | V | | | | | | | F | | | |
| | p3-11 | V | | | | | | | F | | | |
| | p3-5 | | | | | | | | F | | | |
| | p3-14 | | | | | | | | F | | | |
| | p1-5 | | | | | | | | F | | | |
| | p1-28 | | | | | | | | F | | | |
| | p3-1 | | | | | | | | F | | | |
| | p1-1 | | | | | | | | F | | | |
| | p1-2 | | | | | | | | F | | | |
| | p1-3 | | | | | | | | F | | | |
| | p3-4 | | | | | | | | F | | | |
| | p3-28 | | | | | | | | F | | | |
| 1 μM | p4-2 | | | | | | | | | | | |
| HCV-796 | p4-3 | | | | | | | | | | | |
| | p4-4 | | | | | | | | | | | |
| | p4-5 | | | | | | | | | | | |
| | p4-7 | | | | | | | | | | | |
| | p4-8 | | | | | | | | | | | |
| | p4-10 | | | | | | | | | | | |
| | PCR4 | | | | | | | | | | | |
| | PCR4M | | | | | | | | | | | |
| | p4-9 | | L | | | | | | | | | |
| | p6-10 | | L | | | | | | | | | |
| | PCR6 | | L/S | | | | | | C/F | | | A/V |
| | p9-8 | | A | | | | | | F | | | |
| | p9-9 | | T | | | | | | F | | | |
| | p6-1 | | | | | | | | F | | | A |
| | p6-2 | | | | | | | | F | | | A |
| | p6-3 | | | | | | | | F | | | A |
| | p6-11 | | | | | | | | F | | | A |
| | p6-26 | | | | | | | | F | | | A |
| | p6-8 | | | | | | | | F | | | A |
| | p6-12 | | | | | | | | F | | | |
| | p9-2 | | | | | | | | F | | | |
| | p9-13 | | | | | | | | F | | | |
| | p9-1 | | | | | | | | F | | | |
| | p9-3 | | | | | | | | F | | | |
| | p9-4 | | | | | | | | F | | | |
| | p9-7 | | | | | | | | F | | | |
| | PCR9 | | | | | | | | F | | | |
| | p9-5 | V | | | | | | | F | | | |
| | p9-6 | V | | | | | | | F | | | |
| | p4-14 | | L | | | | | | | | | |
| | p6-4 | | L. | | | | | | | | | |
| | p6-7 | | L | | | | | | | | | |
| | p9-14 | | | | | | | | F | | | |
| 10 μM | p41-1 | | | | | I | | T | | | | |
| HCV-796 | p41-2 | | | | | I | | T | | | | |
| | p41-3 | | | | | I | | | | | | |
| | p41-5 | | | | | I | | | | | | |
| | p41-15 | | | | | I | | | | | | |
| | PCR41 | | | | | I | | | | | | |
| | p41-7 | | | | | I | G | | | | | |
| | p41-17 | | | | | I | | | | | | |
| | p49-3 | | | | | | | | F | | | |
| | p41-4 | | | | | | | | F | | | |
| | p41-6 | | | | | | | | F | | | |
| | p44-14 | | | | | | | | | | | |
| | p44-15 | | | | | | | | | | | |
| | p44-20 | | | | | | | | | | | |
| | p44-32 | | | | | | | | | | | |
| | p44-16 | | | | | | | | | R | | |
| | p44-23 | | | | | | | | | | | |
| | p44-13 | | | | | | | | | | | |
| | PCR44 | | L/S | | | | | | | | | |
| | p46-15 | | L | F | I | | | | | | | |
| | p46-16 | | L | F | I | | | | | | | |
| | p49-5 | | L | | | | | | F | | | |
| | PCR46 | | L | | I | | | | | | | |
| | p46-7 | | L | | I | | | | | | | |
| | p46-14 | | L | | I | | | | | | | |
| | p46-8 | | L | | I | T | | | | | | |
| | p46-12 | | L | | I | | G | | | | | |
| | p46-9 | | L | | I | | | | | | | |
| | p46-11 | | L | | I | | | | | | | |
| | p46-13 | | L | | I | | | | | | | |
| | p44-18 | | L | | | | | | F | R | | |
| | p41-13 | | T | | | | | | F | | | |
| | p49-1 | | T | | | | | | F | | | |
| | p49-4 | | T | | | | | | F | | | |
| | p49-2 | | A | | | | | | F | | | |
| | PCR49 | | L/S | | | | | | F | | | |
| | p44-12 | | L | | | | | | | | | |
| | p44-22 | | L | | | | | | | | | |
| | p46-10 | | L | | | | | | | | | |

| | | | | | | | | | | | |
|---|---|---|---|---|---|---|---|---|---|---|---|
| No. of clones out of | 4 | 29 | 2 | 10 | 13 | 2 | 2 | 50 | 2 | 5 | 7 |
| 93 clones | | | | | | | | | | | |
| Frequency of amino | 4.3 | 31.2 | 2.2 | 10.8 | 14.0 | 2.2 | 2.2 | 53.8 | 2.2 | 5.4 | 7.5 |
| acid substitution | | | | | | | | | | | |

TABLE 3
Combination of Amino Acid Substitutions in Replicon Variants

| Combination of amino acid | Number of Clones | Frequency of |
|---|---|---|
| substitutions^{a} | (out of 93 clones) | Mutation (%) |
| K355R and C445F¹ | 12 | 13.0 |
| V85L. F162Y and C316F² | 17 | 18.3 |
| V85L, F162Y, C316F and | 9 | 9.7 |
| T19P² | | |
| C316S/Y and C445F² | 9 | 9.7 |
| P197A, C445F and V581A³ | 8 | 8.6 |
| C316Y and M414I⁴ | 7 | 7.5 |
| S365L and T390I⁴ | 10 | 11.0 |

^{a}NS5B gene was amplified and sequenced from resistant replicon pools selected from:
¹0.1, 1 and 10 μM HCV-796;
²1 and 10 μM HCV-796;
³1 μM HCV-796 and
⁴10 μM HCV-796.

TABLE 4
Activity of HCV-796 Against HCV-796 Replicon Variants

| | | | | Viral Load | |
|---|---|---|---|---|---|
| Replicon | HCV RNA | | Fold | (HCV copies/μg) ± | Viral Load |
| Variant^{a} | EC₅₀ (nM) ± SD^{b} | | Resistance | SD | Reduction^{c} |
| 1b, BB7 | 3.0 ± 1.0 | (n = 11) | — | 1.8 ± 1.1 × 10⁸ | 1.9 ± 0.3 |
| 1b, BB7-L314F | 4 ± 2 | (n = 4) | 1 | 0.3 ± 0.1 × 10⁸ | 1.6 ± 0.3 |
| 1b, BB7-C316F | 392 ± 209 | (n = 4) | 130 | 1.0 ± 0.2 × 10⁸ | 0.8 ± 0.5 |
| 1b, BB7-C316Y | 501 ± 291 | (n = 4) | 166 | 1.3 ± 0.6 × 10⁸ | 0.9 ± 0.2 |
| 1b, BB7-C316N^{d} | 220 ± 110 | (n = 4) | 26^{d} | N/A | N/A |
| 1b, BB7-C316S | 30 ± 4 | (n = 4) | 10 | 1.3 ± 0.7 × 10⁸ | 1.3 ± 0.1 |
| 1b, BB7-I363V | 16 ± 5 | (n = 3) | 5 | 0.2 ± 0.1 × 10⁸ | 1.4 ± 0.1 |
| 1b, BB7-S365A | 124 ± 41 | (n = 4) | 41 | 1.2 ± 0.3 × 10⁸ | 1.7 ± 0.1 |
| 1b, BB7-S365T | 643 ± 168 | (n = 4) | 212 | 1.3 ± 0.6 × 10⁸ | 0.6 ± 0.1 |
| 1b, BB7-S365L | N/A^{e} | | N/A^{e} | N/A^{e} | N/A^{e} |
| 1b, BB7-S368F | 5 ± 2 | (n = 4) | 2 | 2.6 ± 1.2 × 10⁸ | 1.4 ± 0.3 |
| 1b, BB7-M414I | 23 ± 3 | (n = 5) | 8 | 1.3 ± 0.5 × 10⁸ | 1.5 ± 0.2 |
| 1b, BB7-M414T | 3 ± 1 | (n = 4) | 1 | 1.5 ± 0.7 × 10⁸ | 2.0 ± 0.2 |
| 1b, BB7-M414V | 8 ± 1 | (n = 3) | 3 | 0.4 ± 0.1 × 10⁸ | 1.5 ± 0.1 |

^{a}1b, BB7 represents HCV genotype 1b, BB7 isolate. The nomenclature of the replicon NS5B variants (e.g., L314F) is expressed as the amino acid of the input replicon, amino acid position and amino acid substitution.
^{b}EC₅₀ values were determined using the MDL LSW data analysis ™. Inhibitory activity is expressed as mean EC₅₀ ± standard deviation. n indicates number of determinations.
^{c}Viral load reduction was determined at 2240 nM HCV-796 in a 3-day assay. Data represent the mean log reduction of viral RNA ± standard deviation. Results represent at least 3 independent determinations.
^{d}The evaluation of 1b, BB7-C316N was evaluated in a separate laboratory. The EC₅₀ for HCV-796 in 1b, BB was 8.6 ± 4 (n = 14), which was used to calculate the fold resistance for 1b, BB7-C316N.
^{e}Replicon variant S365L failed to establish a stable cell line upon selection with G418.

TABLE 5
Colony Formation Efficiency of Replicon Variants in Huh-7 Cells

| Replicon Variant | CFU/μg RNA |
|---|---|
| 1b, BB7 control | 20,000 |
| L314F | 1500 |
| C316F | 3,000-5,000 |
| C316S | 3,000-5,000 |
| I363V | 100 |
| S365A | 1000 |
| S365T | 120 |
| S365L | 20^{a} |
| M414V | 500 |
| M414T | 3000 |

^{a}did not survive G418 selection

TABLE 6
Activity of HCV-796 on HCV NS5B Enzyme Variants

| | | | Susceptibility Relative to |
|---|---|---|---|
| Enzyme | IC₅₀ (nM) ± SD | | Wild type Enzyme |
| BB7 (C316) | 40 + 20 | (n = 35) | — |
| BB7-C316N | 81 ± 42 | (n = 4) | 2-fold less |
| BB7-C316Y | 320 ± 10 | (n = 3) | 8-fold less |
| BB7-C316F | 1508 ± 419 | (n = 3) | 124-fold less |
| BB7-M414V | 28 + 2 | (n = 3) | 1.4-fold more |
| BB7-M414I | 24 + 6 | (n = 3) | 1.7-fold more |
| BB7-I363V | 60 + 10 | (n = 3) | 1.5-fold less |
| BK (N316) | 140 ± 50 | (n = 33) | — |
| BK-N316C | 31 ± 4 | (n = 3) | 4.5-fold more |

TABLE 7
Activities of Antiviral Agents Against HCV-796-associated Resistant
Replicon Variants

| | | EC₅₀ (μM or pg/ml) ± | | |
|---|---|---|---|---|
| Compound | Replicon | SD | | Fold Resistance |
| PegIFN α-2b^{a} | 1b, BB7 (WT) | 7.0 ± 0.5 | (n = 3) | — |
| | C316Y | 8.0 ± 3.9 | (n = 3) | 1.1 |
| RBV | 1b, BB7 (WT) | 132.6 ± 40.5 | (n = 5) | — |
| | C316Y | 200.9 ± 36.7 | (n = 3) | 1.8 |
| HCV-371 | 1b, BB7 (WT) | 12.2 ± 1.8 | (n = 2) | — |
| | C316Y | 10.8 ± 0.8 | (n = 2) | 0.9 |

^{a}expressed in pg/ml

TABLE 8
Amino Acid Occurrence in Natural HCV Isolates

| | Amino acid | | | | | | |
|---|---|---|---|---|---|---|---|
| Genotype^{a} | 314 | 316 | 363 | 365 | 368 | 414 | 445 |
| 1a (n = 142) | 97% Leu | 100% | 100% Ile | 99% Ser | 100% Ser | 100% | 100% |
| | 3% Val | Cys | | 1% Trp | | Met | Cys |
| 1b (n = 117) | 100% | 40% Asn | 100% Ile | 100% Ser | 100% Ser | 100% | 100% |
| | Leu | 56.5% | | | | Met | Cys |
| | | Cys | | | | | |
| | | 3.5% Tyr | | | | | |
| 2 (n = 13) | 100% | 100% | 100% Ile | 100% Ser | 100% Ser | 100% | 100% |
| | Leu | Cys | | | | Gln | Phe |
| 3b (n = 45) | 100% | 100% | 100% Ile | 100% Ser | 100% Ser | 100% | 100% |
| | Leu | Cys | | | | Met | Phe |
| 4 (n = 22) | 100% | 95.5% | 100% Ile | 100% Ser | 100% Ser | 100% | 100% |
| | Leu | Cys | | | | Val | Phe |
| | | 4.5% Asn | | | | | |
| 5 (n = 2) | 100% | 100% | 100% | 100% Ser | 100% Ser | 100% | 100% |
| | Leu | Cys | Val | | | Met | Phe |
| 6 (n = 2) | 100% | 100% | 100% Ile | 100% Ser | 100% Ser | 100% | 100% |
| | Leu | Cys | | | | Met | Phe |

^{a}n indicates number of full length HCV isolates found in GenBank

1.-10. (canceled)

11. A method of identifying an individual with a decreased likelihood of responding to an anti-Hepatitis C viral therapy, comprising
(a) determining the amino acid sequence or structure of a benzofuran binding pocket of the Hepatitis C RNA-dependent RNA polymerase NS5B in a sample from the individual at a first time point; and
(b) determining the amino acid sequence or structure of the benzofuran binding pocket of the Hepatitis C RNA-dependent RNA polymerase NS5B in a sample from the individual at a second time point,
wherein a change in the amino acid sequence or structure of the benzofuran binding pocket of the Hepatitis C RNA-dependent RNA polymerase NS5B in the sample from the individual at the second time point, in comparison to the amino acid sequence or structure of the benzofuran binding pocket of the Hepatitis C RNA-dependent RNA polymerase NS5B from the individual at the first time point, indicates a decreased likelihood that the individual will respond to an anti-Hepatitis C viral therapy.

12. A method of identifying an individual with a decreased likelihood of responding to an anti-Hepatitis C viral therapy, comprising:
(a) determining the amino acid sequence or structure of benzofuran binding pocket of the Hepatitis C RNA-dependent RNA polymerase NS5B in a sample from the individual; and
(b) comparing the amino acid sequence or structure of the benzofuran binding pocket of the Hepatitis C RNA-dependent RNA polymerase NS5B in the sample from the individual to the amino acid sequence or structure of the benzofuran binding pocket of the Hepatitis C RNA-dependent RNA polymerase NS5B in a reference sample,
wherein a change in the amino acid sequence or structure of the benzofuran binding pocket of the Hepatitis C RNA-dependent RNA polymerase NS5B in the sample from the individual, in comparison to the amino acid sequence or structure of the benzofuran binding pocket of the Hepatitis C RNA-dependent RNA polymerase NS5B in the reference sample, indicates a decreased likelihood that the individual will respond to an anti-Hepatitis C viral therapy.

13. A method for monitoring, diagnosing, or prognosing a treatment-resistant Hepatitis C viral infection in a subject, comprising:
(a) determining the amino acid sequence or structure of a benzofuran-binding pocket of the Hepatitis C RNA-dependent RNA polymerase NS5B in a sample from the subject;
(b) administering a benzofuran compound to the subject; and
(c) determining the amino acid sequence or structure of the benzofuran binding pocket of the Hepatitis C RNA-dependent RNA polymerase NS5B in a sample from the subject following administration of the benzofuran to the subject,
wherein a change in the amino acid sequence or structure of the benzofuran binding pocket of the Hepatitis C RNA-dependent RNA polymerase NS5B in a sample from the subject following administration of the benzofuran, in comparison to the amino acid sequence or structure of the benzofuran binding pocket of the Hepatitis C RNA-dependent RNA polymerase NS5B in a sample from the subject prior to administration of the benzofuran, provides a negative indication of the effect of the treatment of the Hepatitis C viral infection in the subject.

14. A method for monitoring the course of treatment of a Hepatitis C viral infection in a subject, comprising:
(a) determining the amino acid sequence or structure of a benzofuran binding pocket of the Hepatitis C RNA-dependent RNA polymerase NS5B in a sample from the subject;
(b) administering a benzofuran to the subject; and
(c) determining the amino acid sequence or structure of the benzofuran binding pocket of the Hepatitis C RNA-dependent RNA polymerase NS5B in a sample from the subject following administration of the benzofuran to the subject,
wherein a change in the amino acid sequence or structure of the benzofuran binding pocket of the Hepatitis C RNA-dependent RNA polymerase NS5B in a sample from the subject following administration of the benzofuran, in comparison to the amino acid sequence or structure of the benzofuran binding pocket of the Hepatitis C RNA-dependent RNA polymerase NS5B in a sample from the subject prior to administration of the benzofuran, provides a negative indication of the effect of the treatment of the Hepatitis C viral infection in the subject.

15. A method for monitoring the course of treatment of a Hepatitis C viral infection in a subject, comprising:
(a) determining the amino acid sequence or structure of a benzofuran binding pocket of the Hepatitis C RNA-dependent RNA polymerase NS5B in a sample from the subject;
(b) administering a benzofuran and at least one additional anti-Hepatitis C agent to the subject; and
(c) determining the amino acid sequence or structure of the benzofuran binding pocket of the Hepatitis C RNA-dependent RNA polymerase NS5B in a sample from the subject following administration of the benzofuran and at least one additional anti-Hepatitis C agent to the subject,
wherein a change in the amino acid sequence or structure of the benzofuran binding pocket of the Hepatitis C RNA-dependent RNA polymerase NS5B in a sample from the subject following administration of the benzofuran and at least one additional anti-Hepatitis C agent, in comparison to the amino acid sequence or structure of the benzofuran binding pocket of the Hepatitis C RNA-dependent RNA polymerase NS5B in a sample from the subject prior to administration of the benzofuran and at least one additional anti-Hepatitis C agent, provides a negative indication of the effect of the treatment of the Hepatitis C viral infection in the subject.

16. A method for prognosing the development of a treatment-resistant Hepatitis C viral infection in a subject, comprising:
(a) determining the amino acid sequence or structure of a benzofuran binding pocket of the Hepatitis C RNA-dependent RNA polymerase NS5B in a sample from the subject at a first time point; and
(b) determining the amino acid sequence or structure of the benzofuran binding pocket of the Hepatitis C RNA-dependent RNA polymerase NS5B in a sample from the subject at a second time point,
wherein a change in the amino acid sequence or structure of the benzofuran binding pocket of the Hepatitis C RNA-dependent RNA polymerase NS5B in the sample from the subject at the second time point, in comparison to the amino acid sequence or structure of the benzofuran binding pocket of the Hepatitis C RNA-dependent RNA polymerase NS5B from the subject at the first time point, indicates an increased likelihood that the subject will develop a treatment-resistant Hepatitis C viral infection.

17. A method for prognosing the development of a treatment-resistant Hepatitis C viral infection in a subject, comprising:
(a) determining the amino acid sequence or structure of a benzofuran binding pocket of the Hepatitis C RNA-dependent RNA polymerase NS5B in a sample from the subject; and
(b) comparing the amino acid sequence or structure of the benzofuran binding pocket of the Hepatitis C RNA-dependent RNA polymerase NS5B in the sample from the subject to the amino acid sequence or structure of the benzofuran binding pocket of the Hepatitis C RNA-dependent RNA polymerase NS5B in a reference sample,
wherein a change in the amino acid sequence or structure of the benzofuran binding pocket of the Hepatitis C RNA-dependent RNA polymerase NS5B in the sample from the subject, in comparison to the amino acid sequence or structure of the benzofuran binding pocket of the Hepatitis C RNA-dependent RNA polymerase NS5B in the reference sample, indicates an increased likelihood that the subject will develop a treatment-resistant Hepatitis C viral infection.

18. A method for monitoring a Hepatitis C viral infection in a subject, comprising:
(a) determining the amino acid sequence or structure of a benzofuran binding pocket of the Hepatitis C RNA-dependent RNA polymerase NS5B in a sample from the subject at a first time point; and
(b) determining the amino acid sequence or structure of the benzofuran binding pocket of the Hepatitis C RNA-dependent RNA polymerase NS5B in a sample from the subject at a second time point,
wherein a change in the amino acid sequence or structure of the benzofuran binding pocket of the Hepatitis C RNA-dependent RNA polymerase NS5B in the sample from the subject at the second time point, in comparison to the amino acid sequence or structure of the benzofuran binding pocket of the Hepatitis C RNA-dependent RNA polymerase NS5B from the subject at the first time point, provides an indication that the Hepatitis C viral infection has changed in severity.

19. A method for monitoring a Hepatitis C viral infection in a subject, comprising:
(a) determining the amino acid sequence or structure of a benzofuran binding pocket of the Hepatitis C RNA-dependent RNA polymerase NS5B in a sample from the subject; and
(b) comparing the amino acid sequence or structure of the benzofuran binding pocket of the Hepatitis C RNA-dependent RNA polymerase NS5B in the sample from the subject to the amino acid sequence or structure of the benzofuran binding pocket of the Hepatitis C RNA-dependent RNA polymerase NS5B in a reference sample,
wherein a change in the amino acid sequence or structure of the benzofuran binding pocket of the Hepatitis C RNA-dependent RNA polymerase NS5B in the sample from the subject, in comparison to the amino acid sequence or structure of the benzofuran binding pocket of the Hepatitis C RNA-dependent RNA polymerase NS5B in the reference sample, provides an indication that the Hepatitis C viral infection has changed in severity.

20. A method for diagnosing the development of a treatment-resistant Hepatitis C viral infection in a subject, comprising:
(a) determining the amino acid sequence or structure of a benzofuran binding pocket of the Hepatitis C RNA-dependent RNA polymerase NS5B in a sample from the subject at a first time point; and
(b) determining the amino acid sequence or structure of the benzofuran binding pocket of the Hepatitis C RNA-dependent RNA polymerase NS5B in a sample from the subject at a second time point,
wherein a change in the amino acid sequence or structure of the benzofuran binding pocket of the Hepatitis C RNA-dependent RNA polymerase NS5B in the sample from the subject at the second time point, in comparison to the amino acid sequence or structure of the benzofuran binding pocket of the Hepatitis C RNA-dependent RNA polymerase NS5B from the subject at the first time point, indicates an increased likelihood that the subject has developed or will develop a treatment-resistant Hepatitis C viral infection.

21. A method for diagnosing the development of a treatment-resistant Hepatitis C viral infection in a subject, comprising:
(a) determining the amino acid sequence or structure of a benzofuran binding pocket of the Hepatitis C RNA-dependent RNA polymerase NS5B in a sample from the subject; and
(b) comparing the amino acid sequence or structure of the benzofuran binding pocket of the Hepatitis C RNA-dependent RNA polymerase NS5B in the sample from the subject to the amino acid sequence or structure of the benzofuran binding pocket of the Hepatitis C RNA-dependent RNA polymerase NS5B in a reference sample,
wherein a change in the amino acid sequence or structure of the benzofuran binding pocket of the Hepatitis C RNA-dependent RNA polymerase NS5B in the sample from the subject, in comparison to the amino acid sequence or structure of the benzofuran binding pocket of the Hepatitis C RNA-dependent RNA polymerase NS5B in the reference sample, indicates an increased likelihood that the subject has developed or will develop a treatment-resistant Hepatitis C viral infection.

22. The method of claim 15, wherein the at least one additional anti-Hepatitis C agent is an immunomodulator.

23. The method of claim 15, wherein the at least one additional anti-Hepatitis C agent is a ribavirin product.

24. The method of claim 11, wherein the benzofuran binding pocket of the Hepatitis C RNA-dependent RNA polymerase NS5B comprises about amino acid residues 120 to 450 of the Hepatitis C RNA-dependent RNA polymerase NS5B.

25. The method of claim 24, wherein the change in the amino acid sequence or structure of the benzofuran binding pocket is an amino acid change selected from the group consisting of those set forth in Table 2B.

26. The method of claim 24, wherein the change in the amino acid sequence or structure of the benzofuran binding pocket occurs at amino acid residue 314, 316, 363, 365, 368, 414 or 445.

27. The method of claim 26, wherein the change in the amino acid sequence or structure of the benzofuran binding pocket is an amino acid change selected from the group consisting of L314F, C316F, C316Y, C316S, C316N, I363V, S365A, S365T, S368F, M414I, and M414V.

28. The method of claim 11, wherein the Hepatitis C RNA-dependent RNA polymerase NS5B is derived from a Hepatitis C virus genotype selected from the group consisting of genotype 1a, genotype 1b, genotype 2, genotype 3, genotype 4, genotype 5, and genotype 6.
